(19) United States
(12) Patent Application Publication
(10) Pub. No.: US 2016/0184601 A1
(43) Pub. Date: Jun. 30, 2016

(54) MAGNETIC STIMULATOR FOR STIMULATING TISSUE WITH A MAGNETIC FIELD

(71) Applicant: TECHNISCHE UNIVERSITÄT MÜNCHEN, München (DE)

(72) Inventors: Bernhard Gleich, Odelzhausen, OT Sittenbach (DE); Nikolai Jung, Muenchen (DE); Volker Mall, Muenchen (DE); Norbert Gattinger, Olching (DE)

(21) Appl. No.: 14/899,950

(22) PCT Filed: Jun. 20, 2014
(86) PCT No.: PCT/EP14/63030

§ 371 Date: Dec. 18, 2015

(30) Foreign Application Priority Data
Jun. 21, 2013 (DE) 10 2013 211 859.7

(51) Int. Cl. A61N 2/02

(52) U.S. Cl. CPC A61N 2/02

(57) ABSTRACT

A magnetic stimulator for stimulation of a tissue by a magnetic field, having a pulse generator device which comprises a pulse capacitor which can be charged by a charging circuit in order to generate a pulse sequence consisting of pulses and having an adjustable repeat rate; and having a programmable control device which adjusts the pulse generator device to generate a complex pulse sequence, which comprises individually configurable pulses, wherein the generated complex pulse sequence is applied to a stimulation coil in order to generate the magnetic field.

The magnetic stimulation can be used for non-invasive investigation and stimulation of tissue, in particular organic tissue. In conjunction with this, an alternating magnetic field is generated by means of a brief flow of current through a coil. Transcranial magnetic stimulation (TMS) is used to stimulate e.g. the human brain by means of the applied alternating magnetic field. By means of the stimulation of e.g. motor areas of the brain, motor evoked potentials (MEPs) in muscle tissue can be deduced, the properties of which and changes in which allow conclusions to be drawn as to the excitability of the areas of the brain under investigation. TMS is principally of significance in the induction and evaluation of cortical plasticity. Cortical plasticity relates to the brain's ability to adapt to changed conditions. Furthermore, repetitive stimulation by means of a pulsed magnetic field can be used during treatment of different conditions, in particular depression. In order to evaluate the corticospinal system, transcranial magnetic stimulation is regularly used for neurological diagnosis owing to its high level of sensitivity and relatively simple implementation. By application of stimulation protocols for transcranial magnetic stimulation, the function of neuronal networks can be both influenced and evaluated.

By means of the alternating magnetic field generated by a stimulation coil, motor neurons of the tissue can be excited to a motor evoked potential and to an accompanying muscle response. This motor evoked potential can be deduced and evaluated. The induced field used for stimulation is generated by means of a pulsed magnetic field, wherein this can be applied to the patient in a contact-free manner and causes no pain whatsoever at that location.

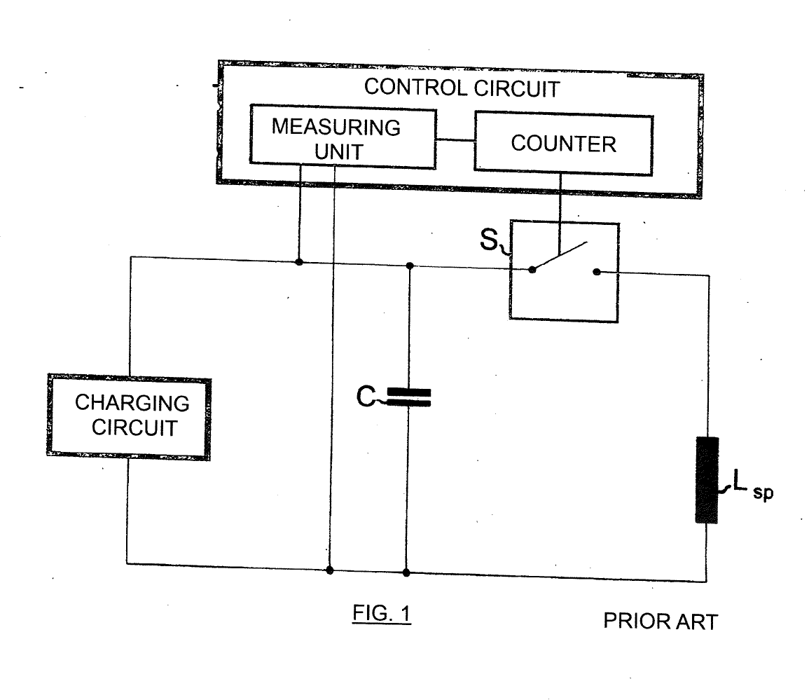

Conventional magnetic stimulators use an oscillating circuit to generate the alternating magnetic field. This oscillating circuit comprises a pulse capacitor and a stimulation coil. FIG. 1 shows a conventional magnetic stimulator as described in DE 10 2006 024 467 A1. This magnetic stimulator contains an oscillation circuit with a pulse capacitor C and a stimulation coil to generate a magnetic field. A charging circuit is provided to charge the pulse capacitor C. Furthermore, the conventional magnetic stimulator in FIG. 1 contains a controllable switch to break and close the oscillation circuit. A control circuit opens and closes the controllable switch such that by means of the oscillation circuit a stimulation pulse with an adjustable number of half or full waves can be generated. The controllable switch can be, for example, a thyristor or an IGBT. With the aid of the controllable switch, integer multiples of full waves can be applied. Prior to pulse triggering, the pulse capacitor is charged to a desired voltage. The energy content of the pulse capacitor sets the current strength through the stimulation coil and therefore the pulse intensity (pulse strength) of the pulse to be output. If the switch is closed, a current begins to flow through the stimulation coil and the pulse capacitor begins to discharge. After the coil current abates, all of the pulse energy is consumed and the pulse capacitor is fully discharged. The pulse capacitor must then be charged to the desired voltage level prior to the next pulse. However, such conventional magnetic stimulators have the disadvantage that the number of pulses generated by the pulse generator device is time-limited. In conventional magnetic stimulators, the maximum repeat rate, i.e. the number of pulses output per unit of time, is 100 pulses per second. A further substantial disadvantage of conventional magnetic stimulators is that they can generate only sinusoidal pulses. Conventional magnetic stimulators generally generate monophase and biphase pulses with adjustable pulse width. Furthermore, with conventional magnetic stimulators only pulse sequences which contain pulses of the same pulse form can be generated. An individual configuration of the pulses with respect to their pulse form and/or pulse polarity in order to create complex pulse sequences is not possible. Individual or flexible adaptation of the generated pulse sequence to the tissue to be investigated or a clinical picture thus cannot be effected with conventional magnetic stimulators.

It is therefore an object of the present invention to create a magnetic stimulator for stimulation of a tissue by a magnetic field, in which the above-mentioned disadvantages are avoided and in which pulse sequences can be adapted flexibly to the tissue to be investigated or to a clinical picture of a patient.

In accordance with the invention, this object is achieved by a magnetic stimulator having the features stated in claim 1.

The invention accordingly creates a magnetic stimulator for stimulation of a tissue by a magnetic field with a pulse generator device which has a pulse capacitor which can be charged by a charging circuit in order to generate a pulse sequence consisting of pulses and having a repeat rate which can be adjusted and having a programmable control device which adjusts the pulse generator device in order to generate a complex pulse sequence which has individually configurable pulses, wherein the generated complex pulse sequence is applied to a stimulation coil in order to generate the magnetic field.

The magnetic stimulator in accordance with the invention makes it possible to generate complex pulse sequences and pulse patterns at a high adjustable repeat rate and to provide a stimulation coil connected to the magnetic stimulator in order to generate the alternating magnetic field. In this way, reproducible and effective changes in plasticity can be achieved in a stimulated brain.

In one possible embodiment of the magnetic stimulator in accordance with the invention, the pulse sequence output by the pulse generator device is a simple pulse sequence consisting of pulses or is a complex pulse sequence.

The generated complex pulse frequency preferably has pulse trains which each comprise pulse packets which each consist of a series of pulses, wherein a pulse form and/or polarity of the pulses is/are individually configurable.

In a further possible embodiment of the magnetic stimulator in accordance with the invention, the programmable control device of the magnetic stimulator can be connected to a computer via an interface, on which computer a user-editor is provided to configure the pulse sequence.

In a further possible embodiment of the magnetic stimulator in accordance with the invention, the user-editor of the computer connected to the magnetic stimulator has a stimulus designer to configure a pulse form of the respective pulses of the pulse sequence.

In a further possible embodiment, the user-editor further comprises a pulse packet assistant to configure at least one pulse packet consisting of pulses.

In a further possible embodiment, the user-editor additionally comprises a pulse train assistant to configure at least one pulse train consisting of pulse packets.

In a further possible embodiment of the magnetic stimulator in accordance with the invention, the complex pulse sequence configured by means of the user-editor is transmitted via the interface to the programmable control device of the magnetic stimulator and is stored in a memory unit of the magnetic stimulator.

In a further possible embodiment of the magnetic stimulator in accordance with the invention, the repeat rate of the pulse sequence, which indicates the number of pulses output per second, can be adjusted within a range of 0 to 1 kHz.

In a further possible embodiment of the magnetic stimulator in accordance with the invention, an evaluation pulse for measuring a motor muscular response of the stimulated tissue is output between pulse packets of the complex pulse sequence which is generated by the pulse generator device of the magnetic stimulator.

In a further possible embodiment of the magnetic stimulator in accordance with the invention, the pulse generator device of the magnetic stimulator has an oscillation circuit, which contains the pulse capacitor and the stimulation coil, and at least one power switch which is connected to a driver circuit which can be controlled by the programmable control device of the magnetic stimulator.

In one possible embodiment of the magnetic stimulator in accordance with the invention, the stimulation coil is in a full bridge circuit connection with four power switches to generate pulses, the pulse form of which can be composed of pulse segments.

In a further possible embodiment of the magnetic stimulator in accordance with the invention, the pulse generator device of the magnetic stimulator has a charging circuit for recharging the pulse capacitor with the adjusted repeat rate.

In one possible embodiment of the magnetic stimulator in accordance with the invention, the charging circuit of the pulse generator device is a linear charging circuit.

In one possible embodiment, this linear charging circuit has a mains adapter for connection to a power supply network, an intermediate energy circuit for intermediate storage of the electrical energy supplied by the mains adapter, and a charge regulator which is connected to the oscillation circuit of the pulse generator device.

In a further possible alternative embodiment of the magnetic stimulator in accordance with the invention, the charging circuit of the pulse generator device has a clocked charging circuit.

In one possible embodiment of the clocked charging circuit, this charging circuit has a mains adapter for connection to a power supply network,

a first DC/DC switching regulator for continuous operation,

an intermediate energy circuit for intermediate storage of the electrical energy supplied from the first DC/DC switching regulator and

a second DC/DC switching regulator for pulsed operation, which is connected to the oscillation circuit of the pulse generator device.

In a further possible embodiment of the magnetic stimulator in accordance with the invention, the pulse generator device has a coil monitoring circuit.

In one possible embodiment of the coil monitoring circuit, this coil monitoring circuit monitors whether a stimulation coil is connected to the magnetic stimulator.

In a further possible embodiment of the magnetic stimulator in accordance with the invention, the coil monitoring circuit has sensors to monitor operating parameters of the stimulation coil, in particular the operating temperature thereof.

In a further possible embodiment of the magnetic stimulator in accordance with the invention, the programmable control device causes the pulse generator device to output the pulse sequence to the stimulation coil only after a system check of parameters of the magnetic stimulator has been successfully concluded.

In a further possible embodiment of the magnetic stimulator in accordance with the invention, the programmable control device can be connected to a conducting electrode which is attached to the tissue to be stimulated, in order to conduct a measurement signal and/or to generate a trigger signal.

In a further possible embodiment of the magnetic stimulator in accordance with the invention, the measurement signal conducted through the conducting electrode is evaluated by the programmable control device in order to determine a motor threshold.

The invention further provides a method for generating a magnetic field having the features stated in claim 17.

According to a further aspect a method for generating a magnetic field is provided having the following steps:

generating a complex pulse sequence which consists of individually configured pulses with a variable pulse form, by means of a pulse generator device,

applying the generated pulse sequence with an adjustable repeat rate to a stimulation coil which generates the magnetic field therefrom and

recharging a pulse capacitor of the pulse generator device by a charging circuit with the adjusted repeat rate.

In one possible embodiment of the method the repeat rate, which indicates the number of pulses per unit of time, is adjusted in a range of 0 to 1 kHz.

In one possible embodiment of the method the generated complex pulse sequence comprises pulse trains which each comprise pulse packets which each consist of a series of pulses, the pulse form and/or polarity of which is/are individually configured.

According to a further aspect a device for use in a method for stimulating a tissue by a magnetic field is provided,

wherein a complex pulse sequence, which consists of individually configured pulses with a variable pulse form, is generated by a pulse generator device,

wherein the generated pulse sequence is applied with an adjustable repeat rate to a stimulation coil which generates the magnetic field therefrom,

wherein a pulse capacitor of the pulse generator device is recharged by a charging circuit with the adjusted repeat rate.

Possible embodiments of the magnetic stimulator in accordance with the invention for stimulation of a tissue by a magnetic field are explained in more detail hereinunder with reference to the attached figures, in which:

FIG. 1 is a block circuit diagram of a conventional magnetic stimulator in accordance with the prior art;

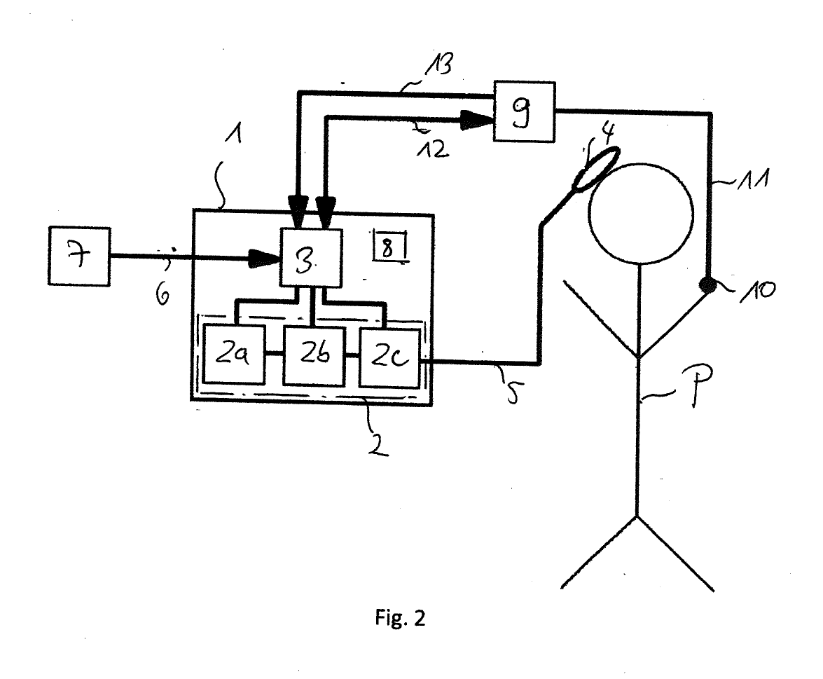

FIG. 2 is a block circuit diagram to illustrate a possible embodiment of a magnetic stimulator in accordance with the invention for stimulation of a tissue by a magnetic field;

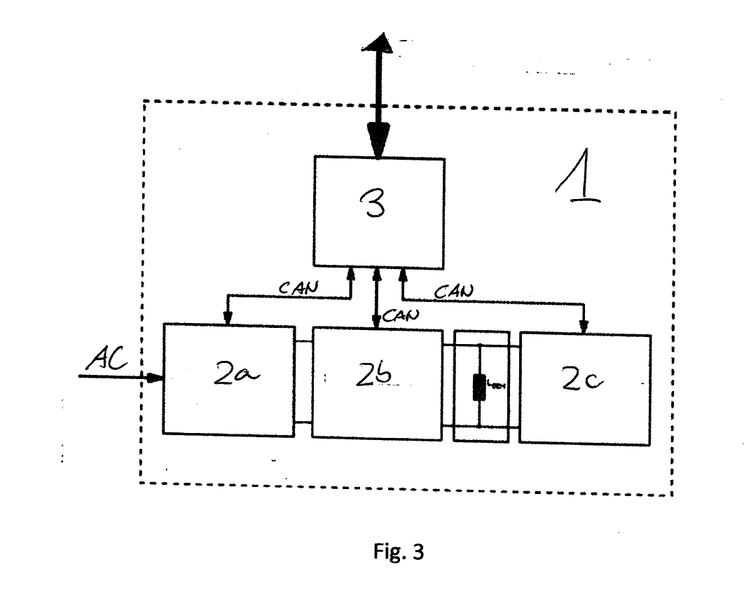

FIG. 3 is a further block circuit diagram to illustrate an exemplified embodiment of the magnetic stimulator in accordance with the invention;

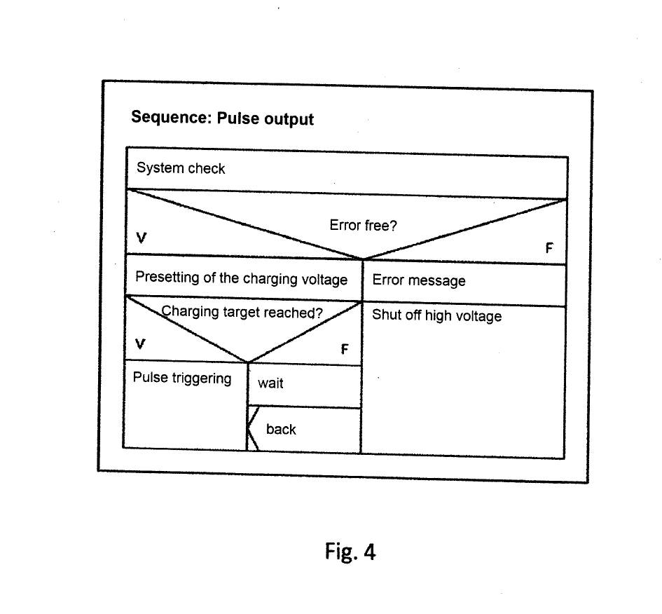

FIG. 4 is a diagram to explain a system check carried out by the control device in the magnetic stimulator in accordance with the invention;

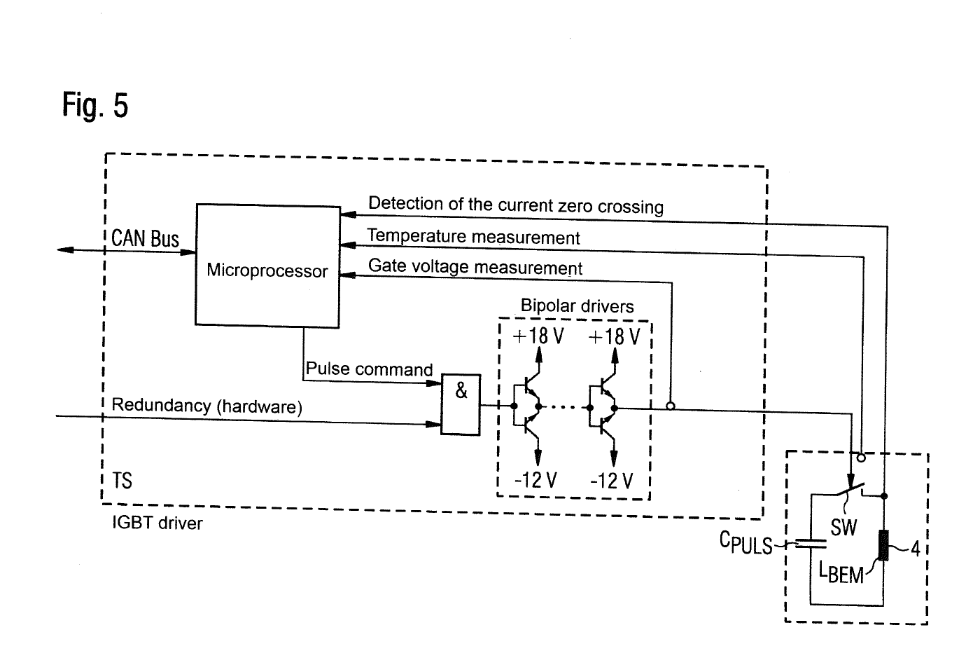

FIG. 5 is a block circuit diagram for illustration of an exemplified embodiment of a driver circuit used in a pulse generator device of the magnetic stimulator in accordance with the invention;

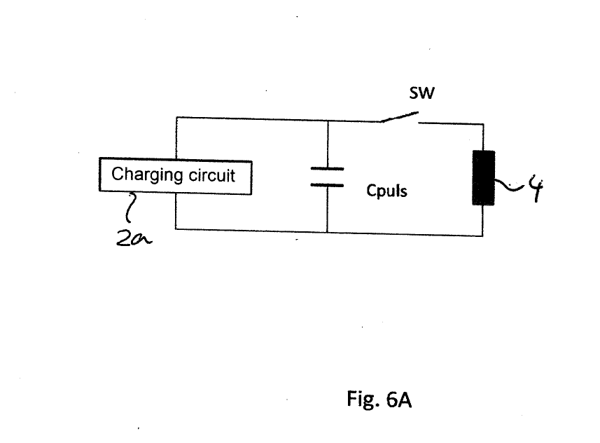

FIG. 6 shows signal diagrams for explanation of current zero crossing identification which is used in the driver circuit used in FIG. 5;

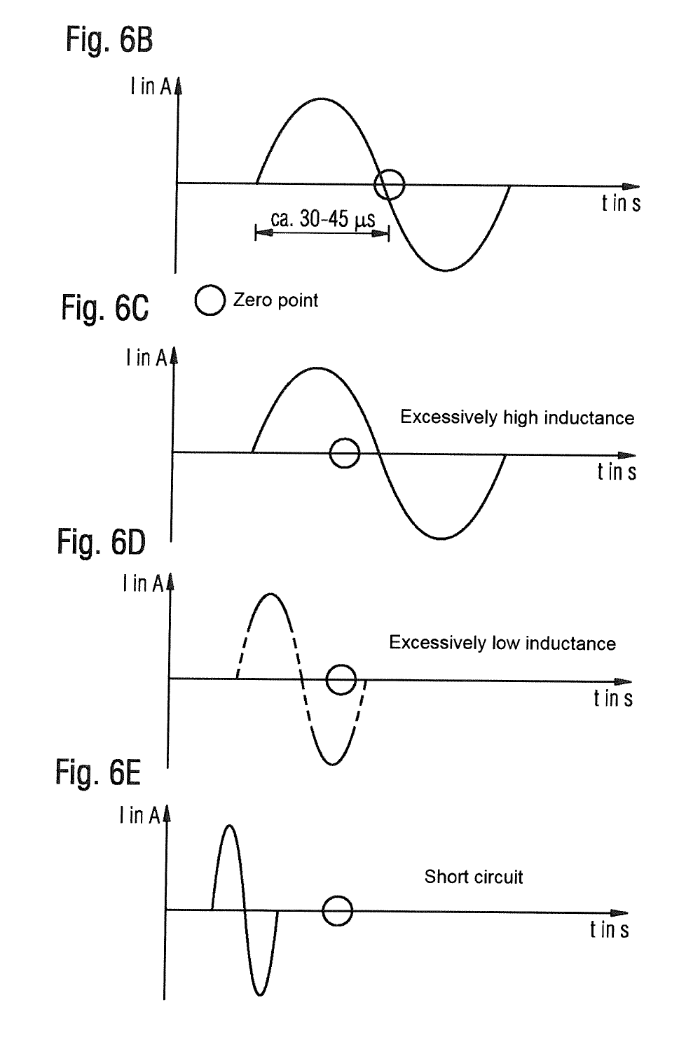

FIG. 7 is a circuit diagram to illustrate an exemplified embodiment of a pulse generator device in which the stimulation coil is in a full bridge circuit connection;

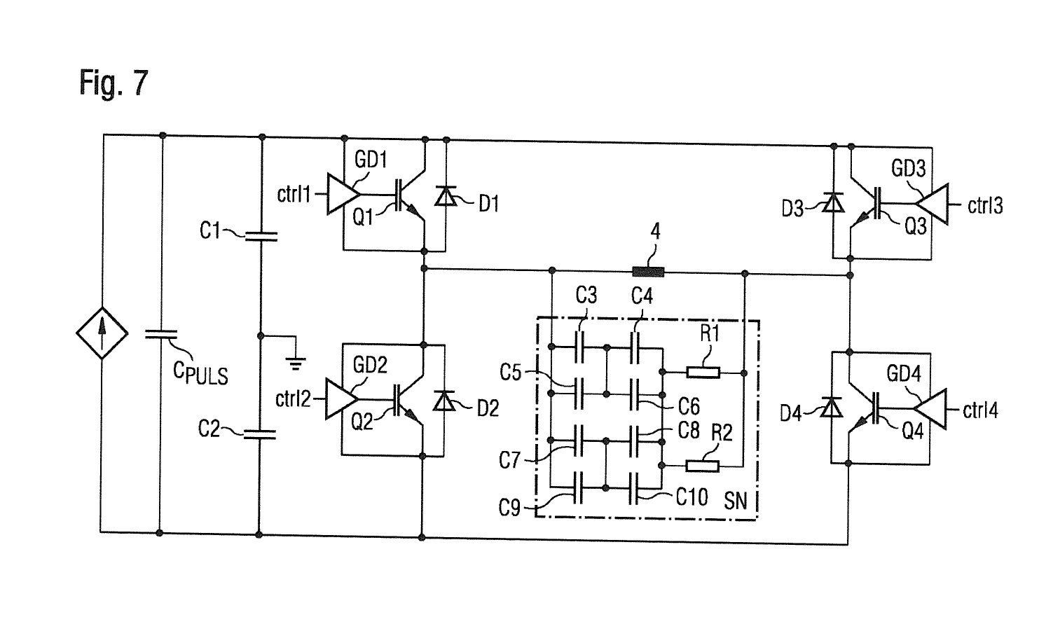

FIG. 8 shows diagrams for explanation of the mode of operation of the full bridge circuit shown in FIG. 7 for generation of pulses from pulse segments;

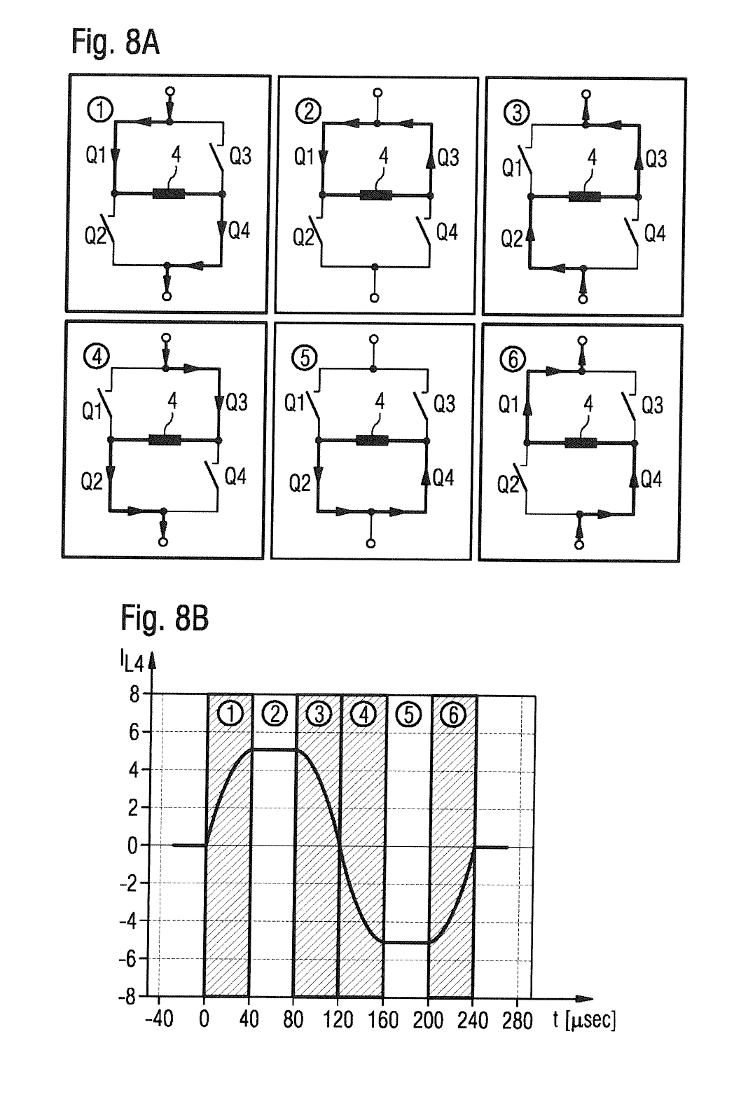

FIG. 9 is a signal diagram for explanation of the actuation of the full bridge circuit shown in FIG. 7 with alternating polarities;

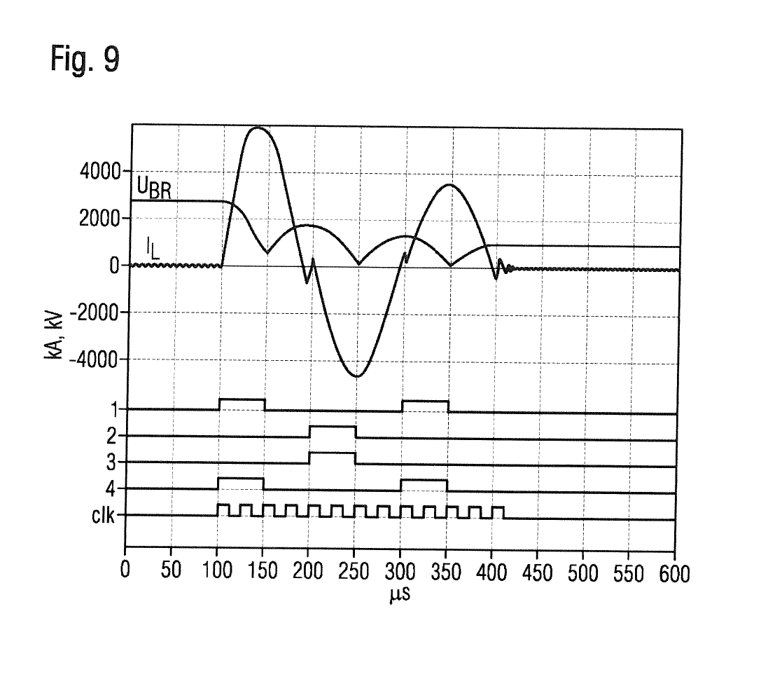

FIG. 10 is a signal diagram for explanation of the actuation of the full bridge circuit shown in FIG. 7 with an individual polarity;

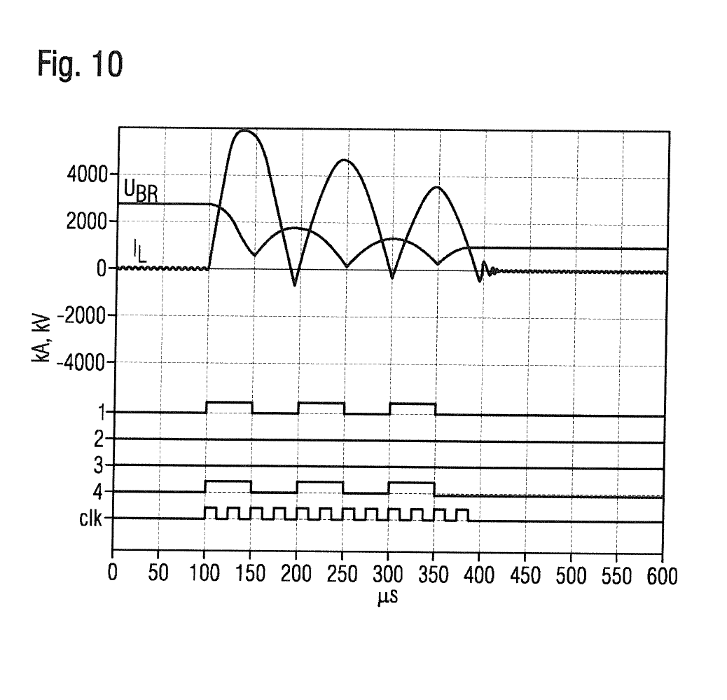

FIG. 11 is a signal diagram for illustration of actuation of the full bridge circuit shown in FIG. 7 with holding phases;

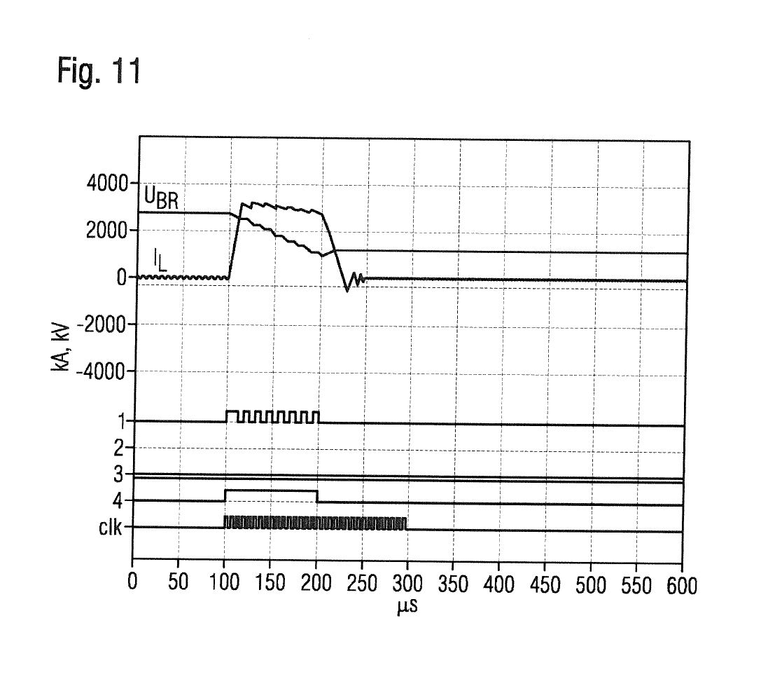

FIG. 12 shows a possible embodiment of a full bridge circuit with switched capacitances;

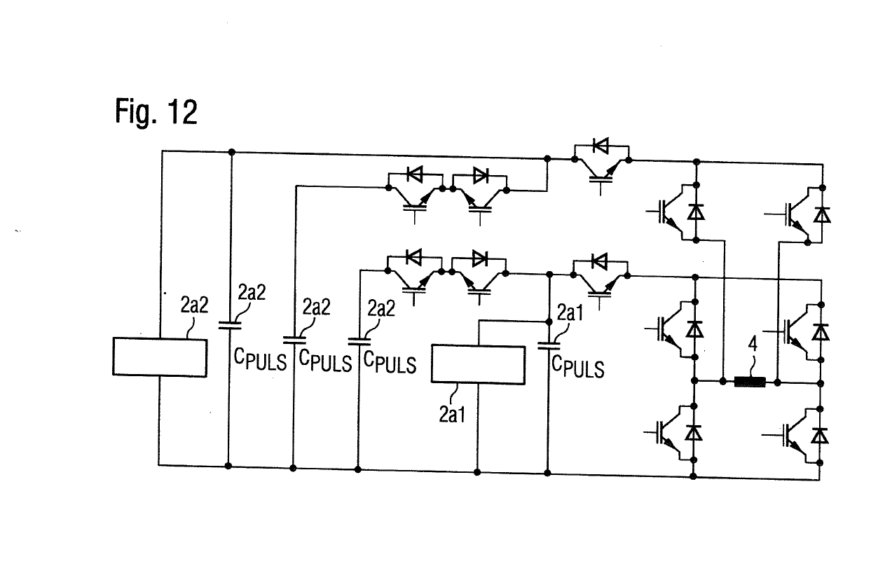

FIG. 13 is a signal diagram for illustration of an exemplified asymmetric pulse form;

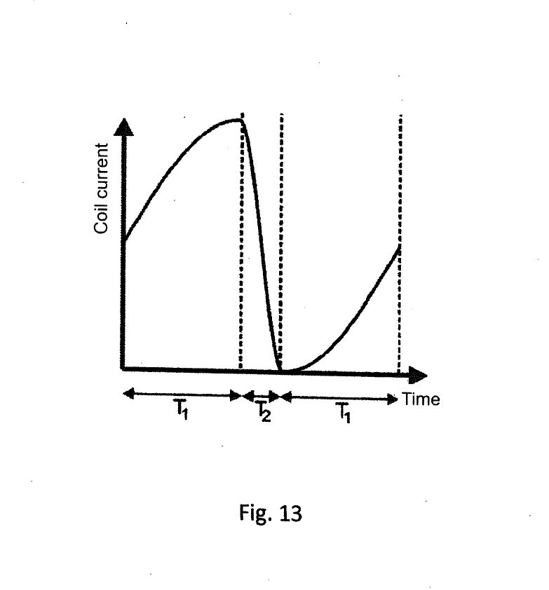

FIG. 14 is a block circuit diagram for illustration of an exemplified embodiment of a charging circuit used within the pulse generator device of the magnetic stimulator;

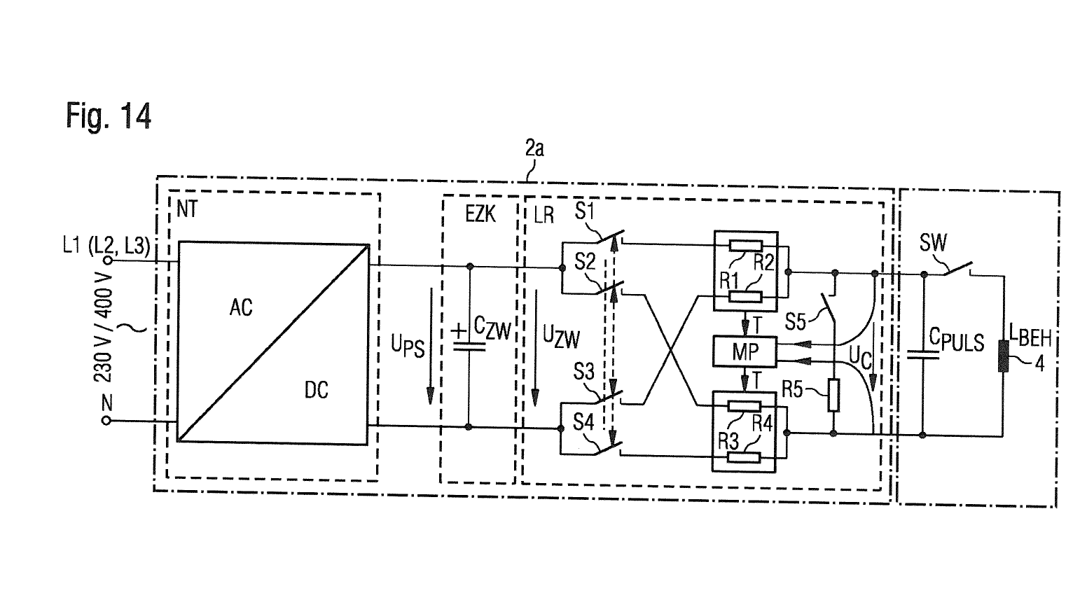

FIG. 15 is a charging curve for explanation of the mode of operation of the intermediate energy circuit used within the charging circuit;

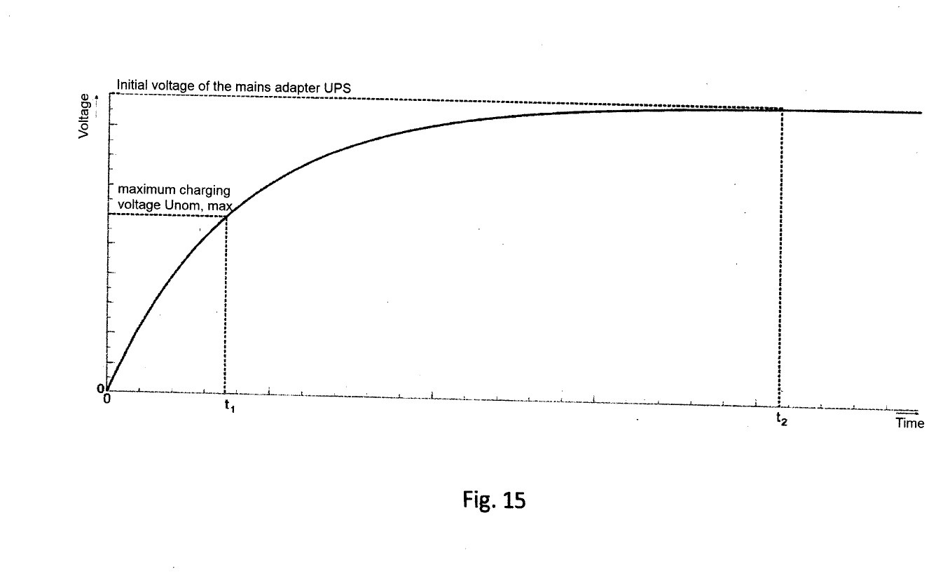

FIG. 16 is a signal diagram for illustration of the voltage progression on a pulse capacitor and for actuation of charging switches of the charging regulation provided within the charging circuit and illustrated in FIG. 14;

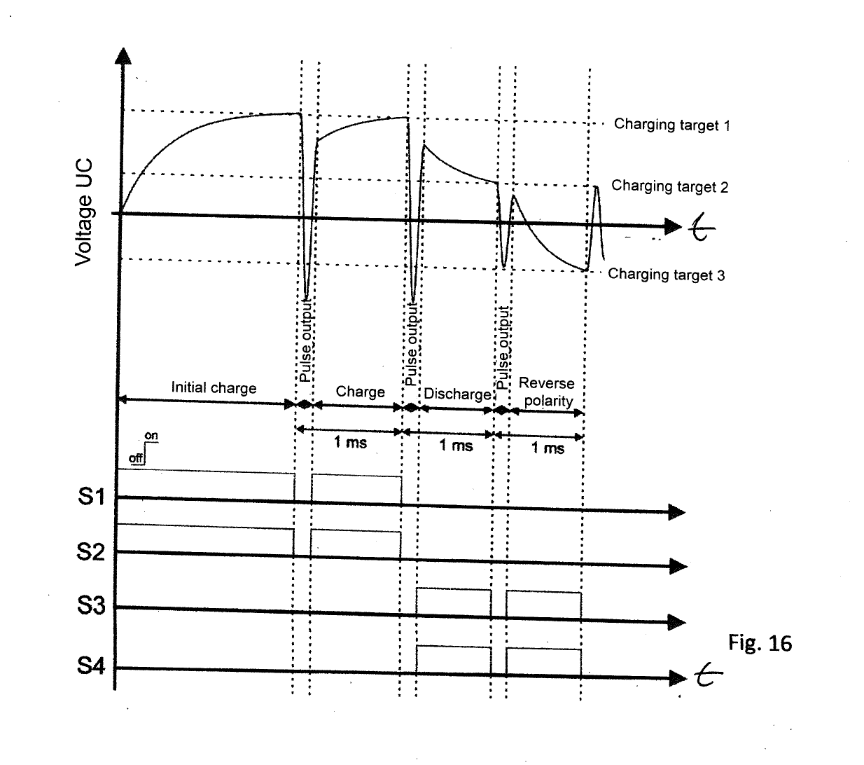

FIG. 17 is a block circuit diagram of a clocked charging circuit used within the pulse generator device of the magnetic stimulator in accordance with the invention;

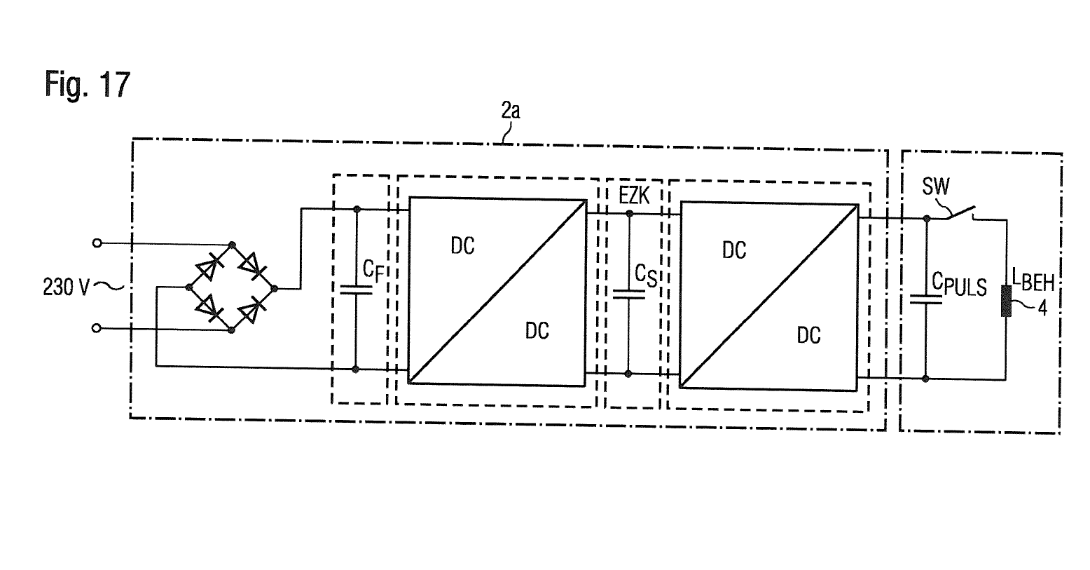

FIG. 18 shows a current progression for explanation of the mode of operation of a particular embodiment of the clocked charging circuit illustrated in FIG. 17;

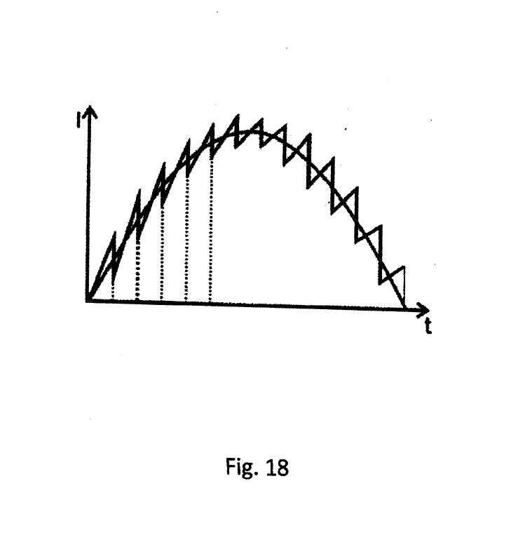

FIG. 19 is a circuit diagram for illustration of an embodiment of a power form correction circuit as an upwards converter;

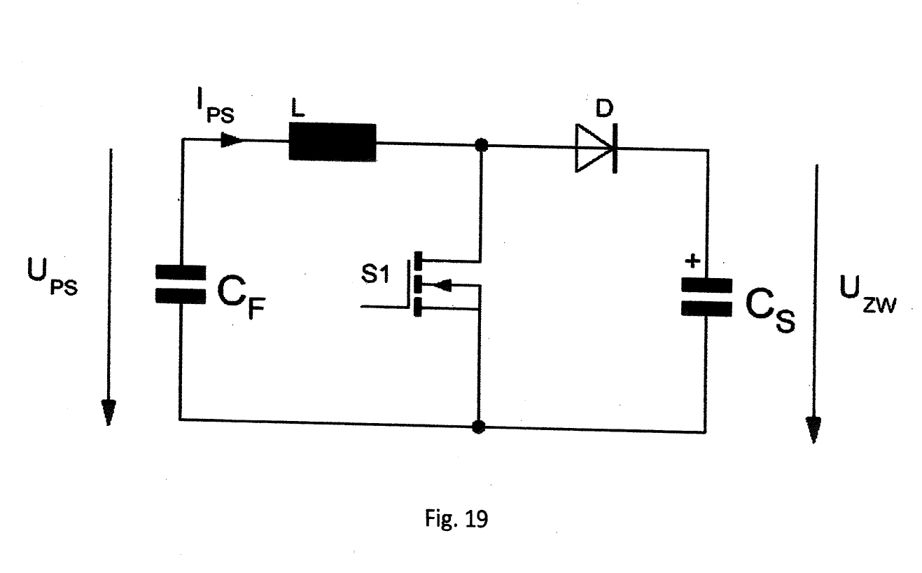

FIG. 20 is a circuit diagram for illustration of an embodiment variation of the charging regulator used in the clocked charging circuit,

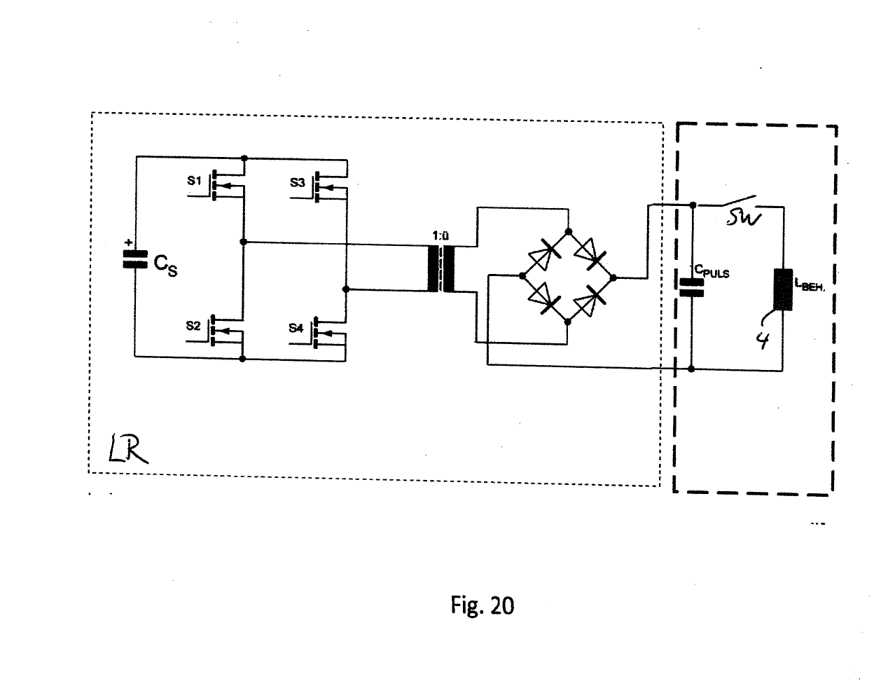

FIG. 21 is a diagram for illustration of a charging current of a pulse capacitor of the embodiment variation of the charging regulator shown in FIG. 20;

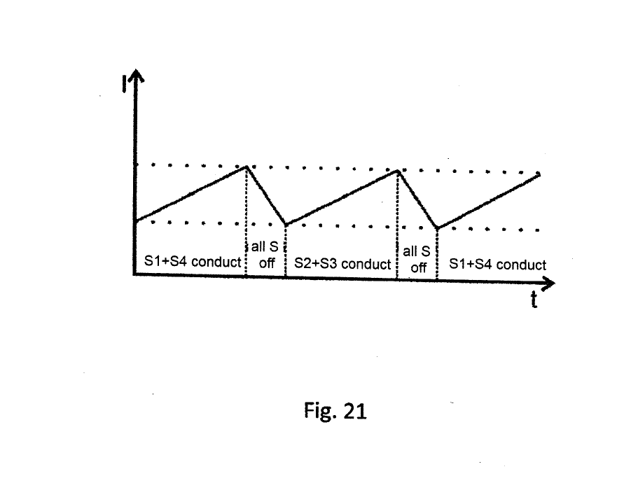

FIG. 22 is a circuit diagram for illustration of a further embodiment variation of the charging regulator which can be used in the clocked charging circuit in accordance with FIG. 17;

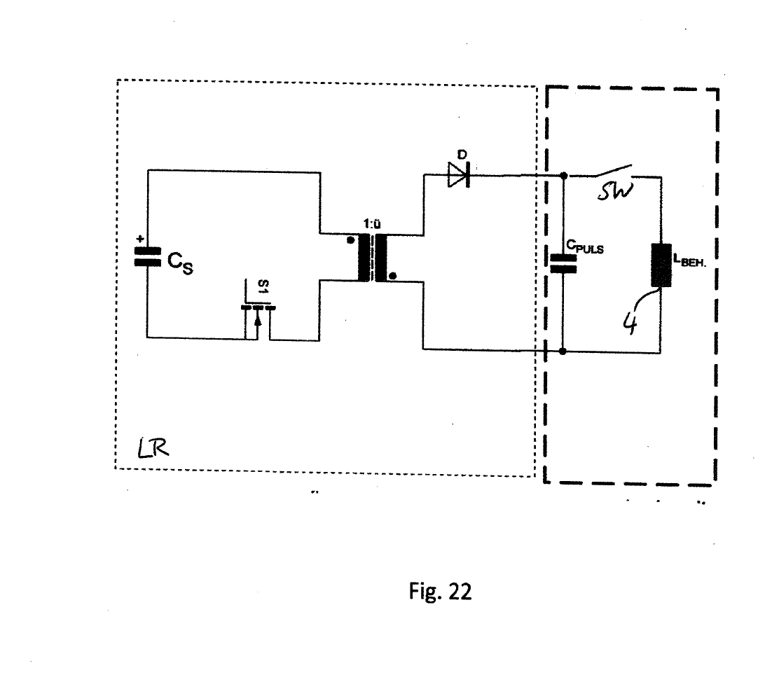

FIG. 23 is a diagram for illustration of the current flow in the variation of a charging regulator shown in FIG. 22;

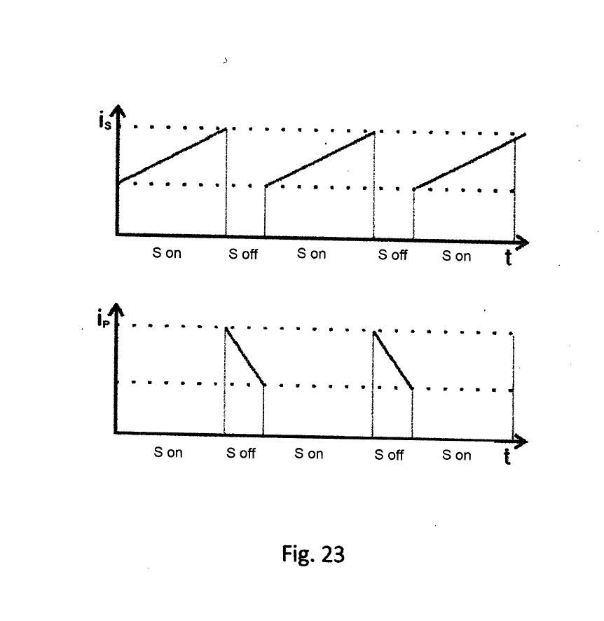

FIG. 24 is a circuit diagram for illustration of a further embodiment variation of a charging regulator as can be used in the clocked charging circuit in accordance with FIG. 17;

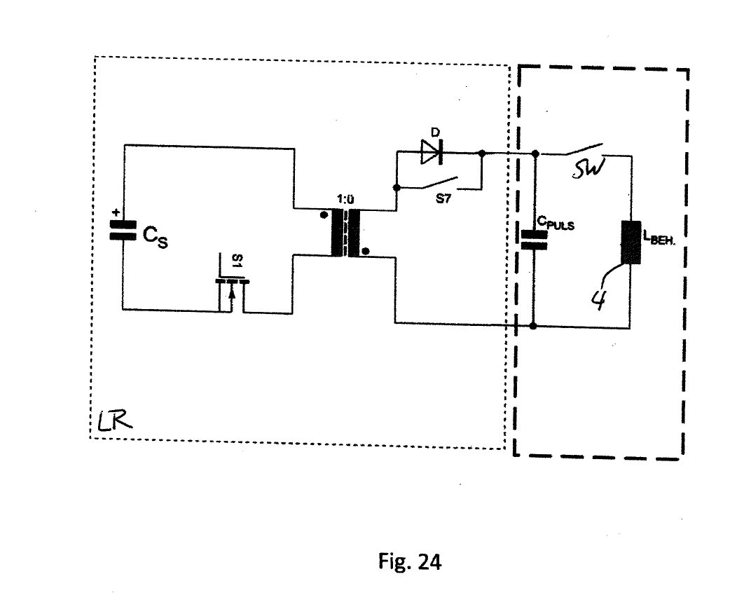

FIG. 25 is a diagram to illustrate a working sequence for configuration of pulse forms of a complex pulse sequence used in the magnetic stimulator in accordance with the invention;

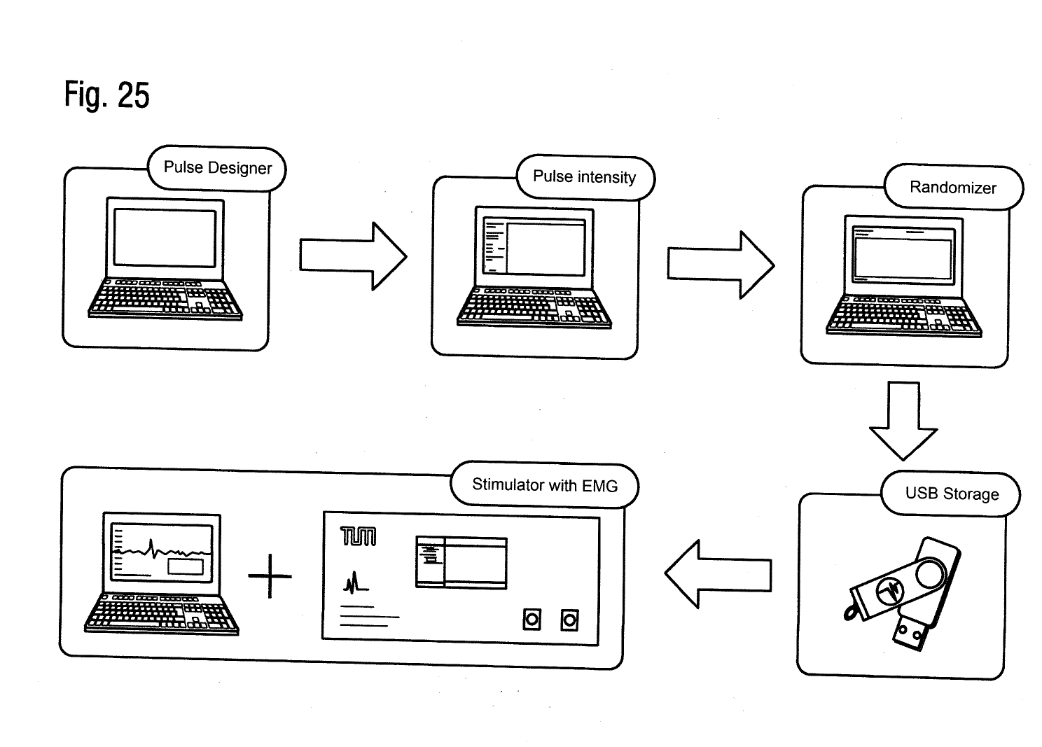

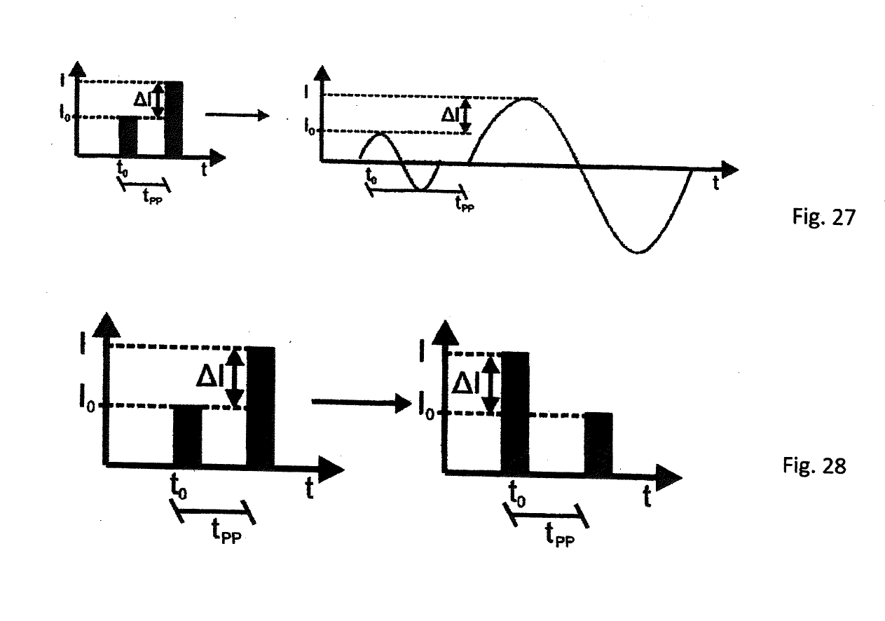

FIGS. 26, 27 and 28 are diagrams to illustrate the pulse variants which can be achieved and which can be contained in a complex pulse sequence of the magnetic stimulator in accordance with the invention;

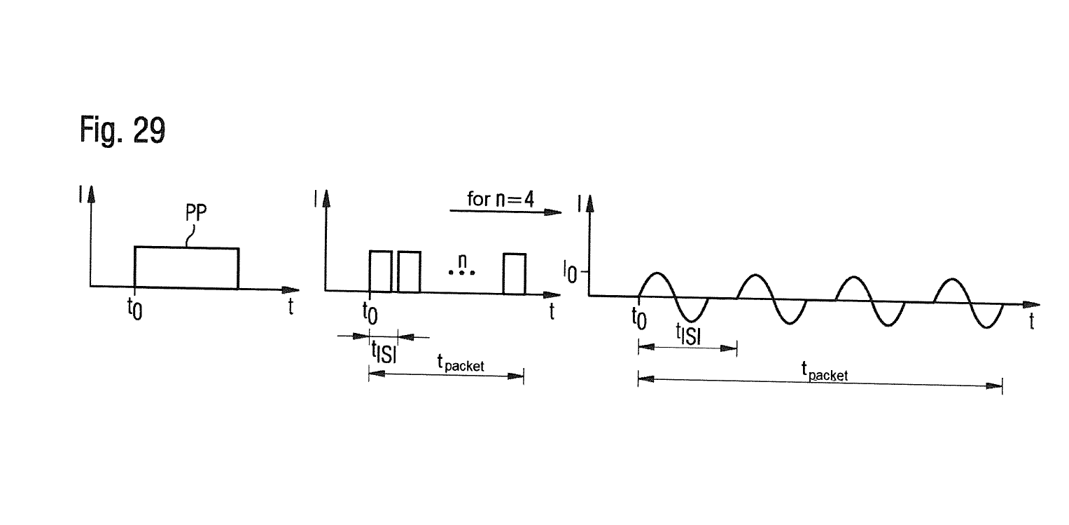

FIG. 29 is a diagram for illustration of a pulse packet within a complex pulse sequence, wherein the pulse packet consists of a preset number of pulses;

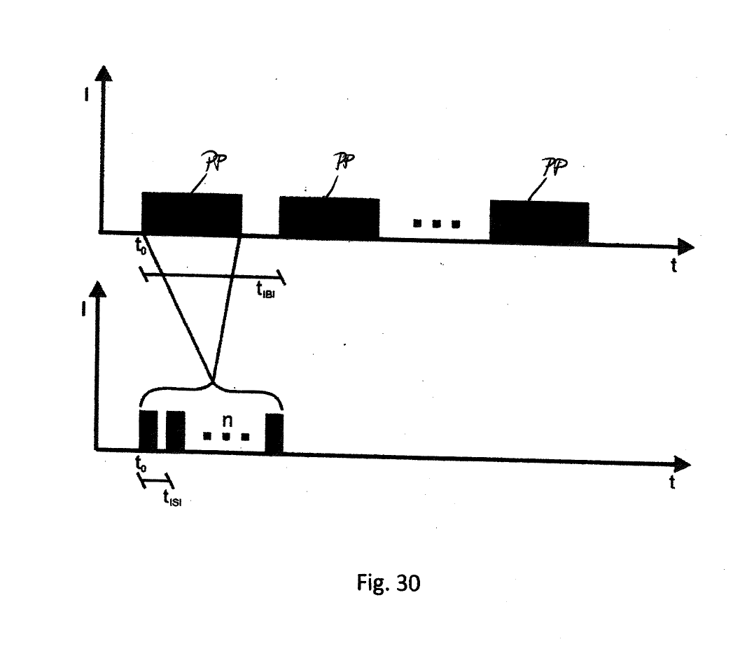

FIG. 30 is a signal diagram for illustration of a plurality of pulse packets which are each composed of individual pulses;

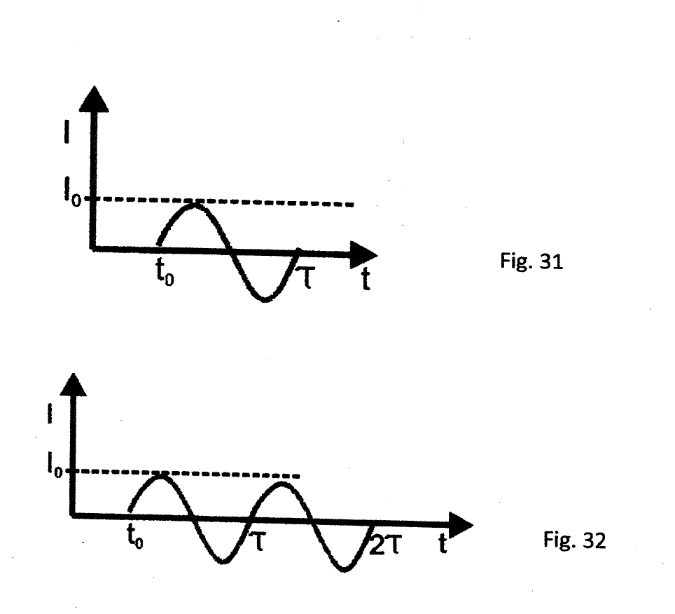

FIG. 31 is a signal diagram to illustrate a single wave as can be contained within a complex pulse sequence of the magnetic stimulator;

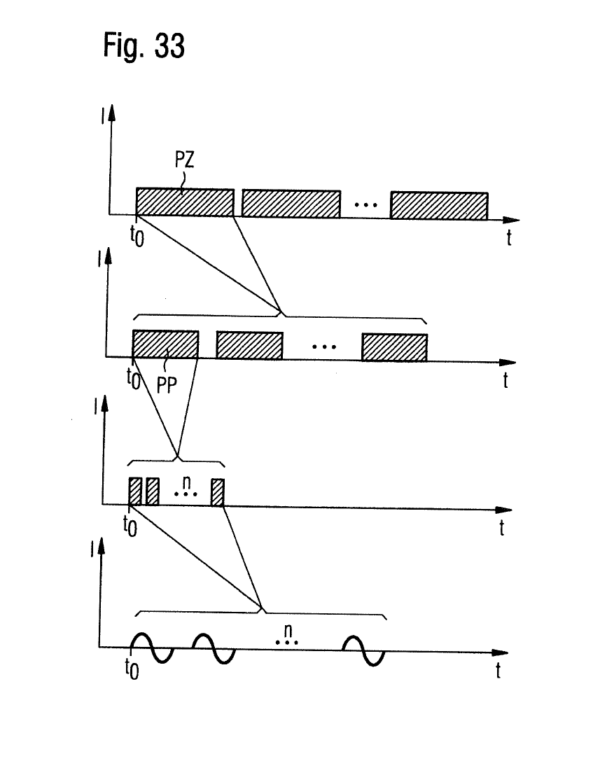

FIG. 32 is a signal diagram to illustrate a double wave as can be contained within a complex pulse sequence of the magnetic stimulator in accordance with the invention;

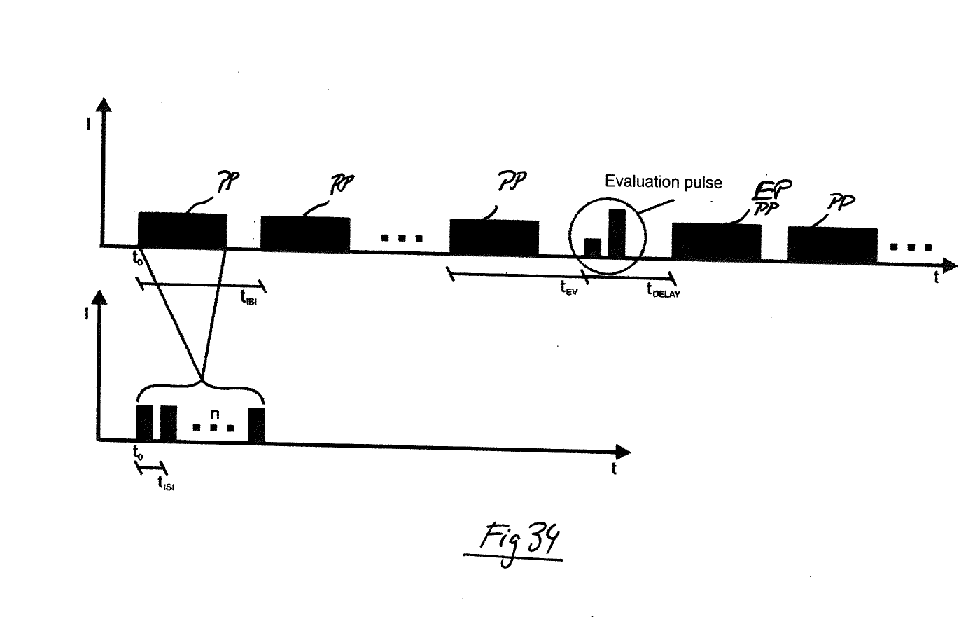

FIG. 33 is a diagram to illustrate a complete complex pulse sequence with a plurality of pulse trains which each consist of pulse packets which are for their part composed of configurable pulses as can be output to a stimulation coil by the magnetic stimulator in accordance with the invention;

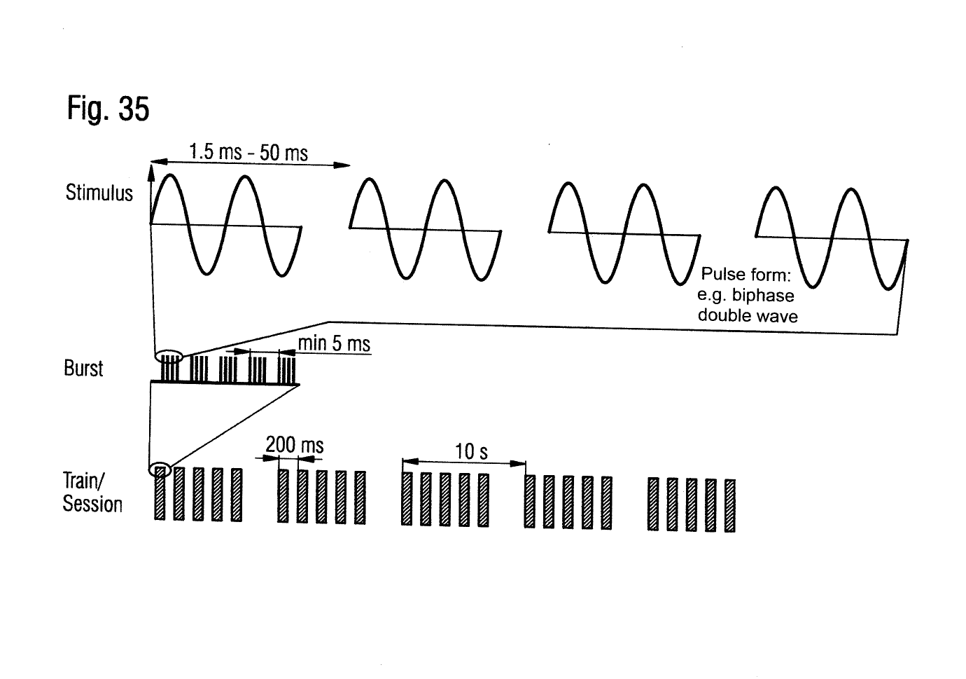

FIG. 34 is a signal diagram to illustrate a complex pulse sequence with an evaluation pulse contained therein for explanation of an embodiment variation of the magnetic stimulator in accordance with the invention;

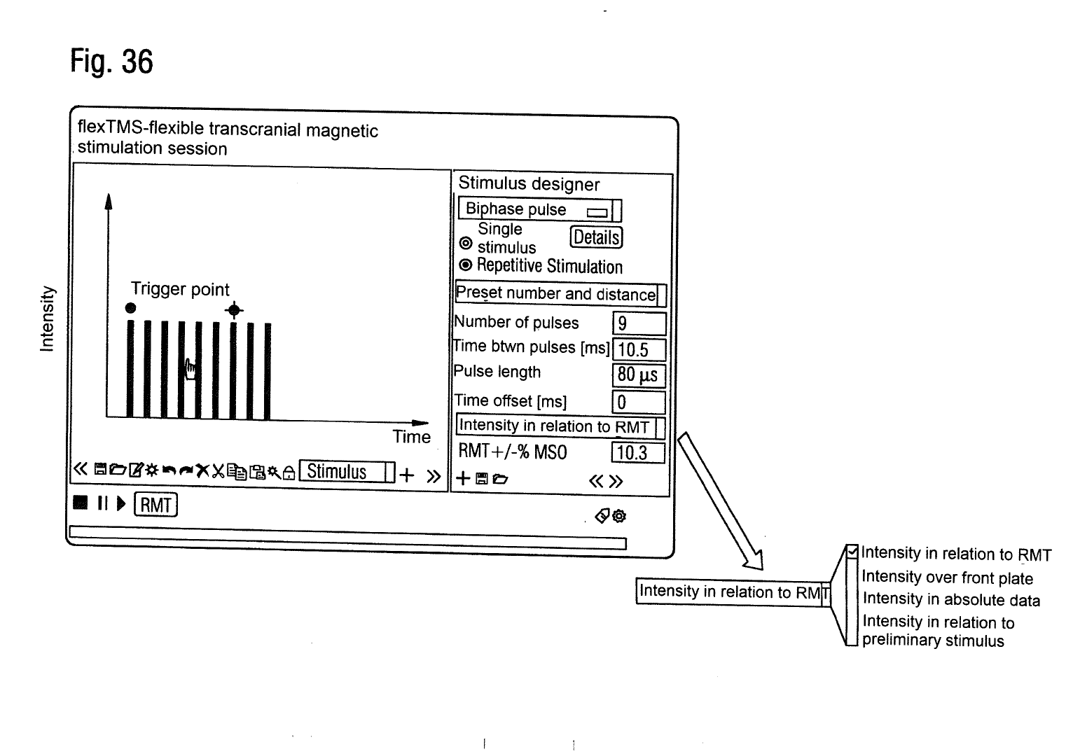

FIG. 35 is a diagram for explanation of the operating sequence of one possible embodiment variation of the magnetic stimulator in accordance with the invention;

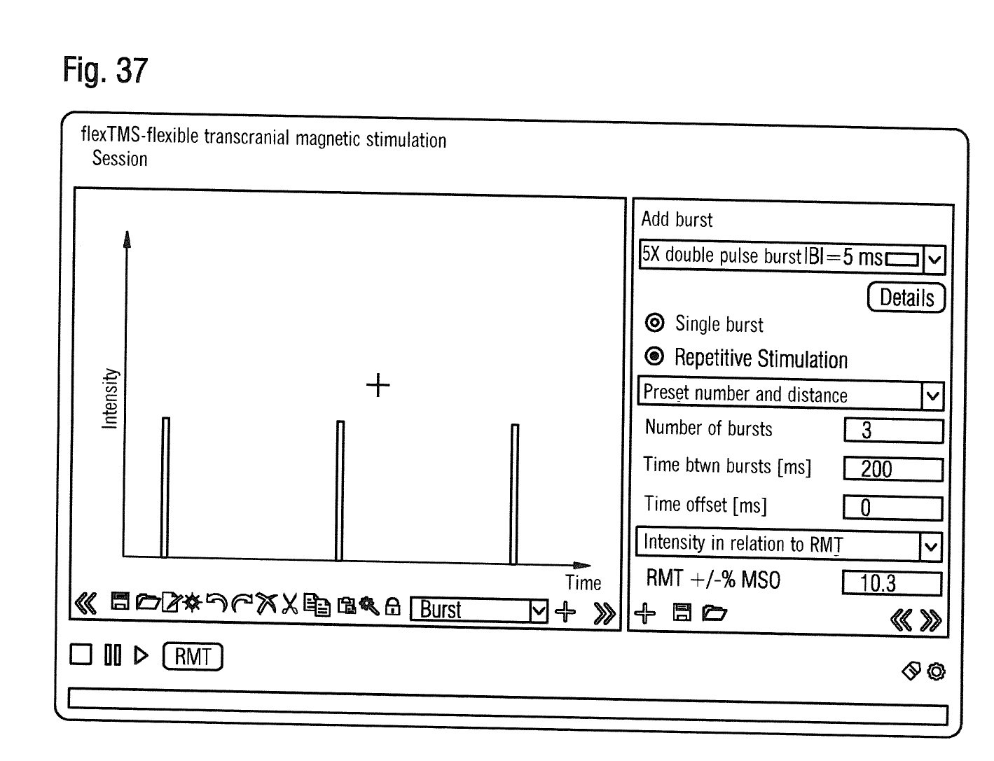

FIG. 36 is a diagram for explanation of an embodiment variation of the user-editor used in the magnetic stimulator in accordance with the invention, having a stimulus designer;

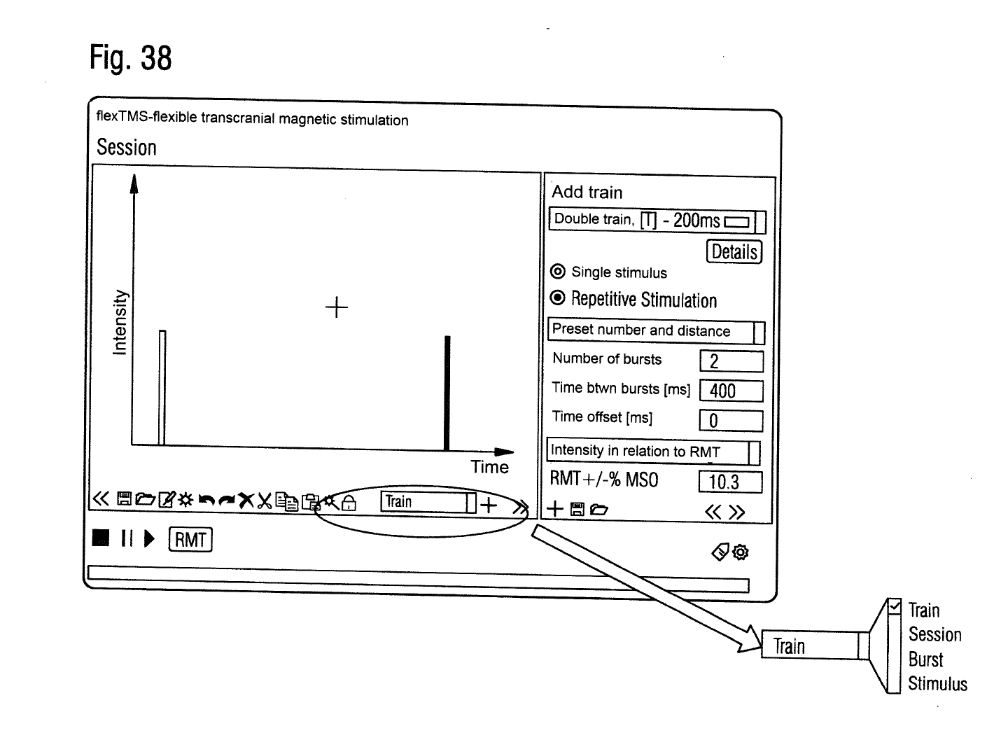

FIG. 37 is an illustration of the pulse packet assistant used in the user-editor;

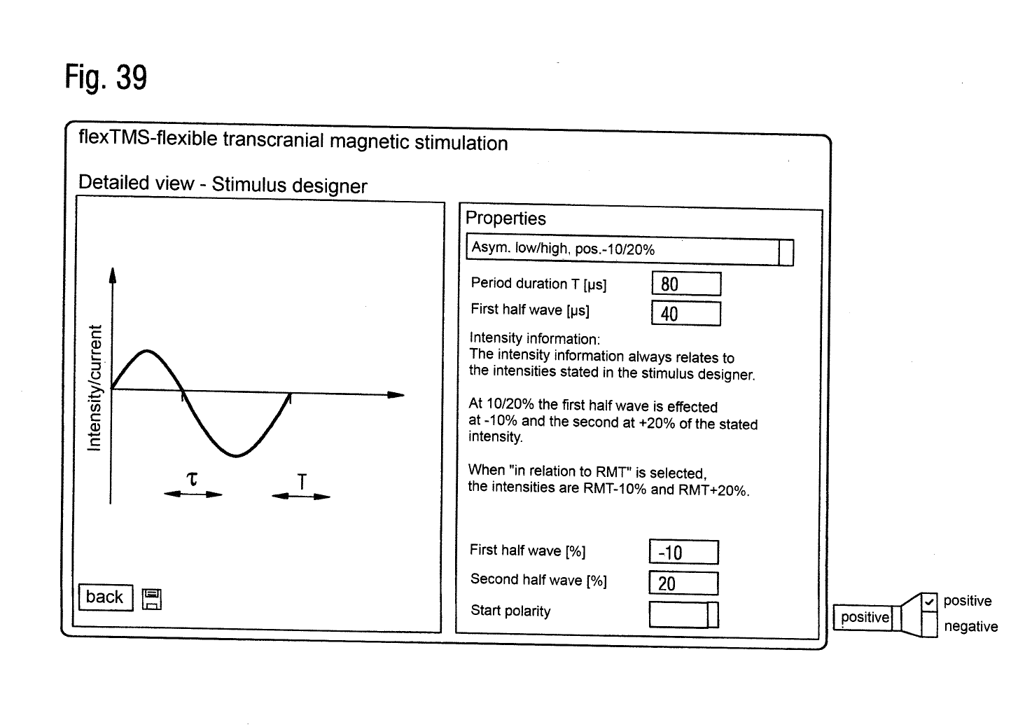

FIG. 38 is a diagram to illustrate a pulse train assistant used in the user-editor;

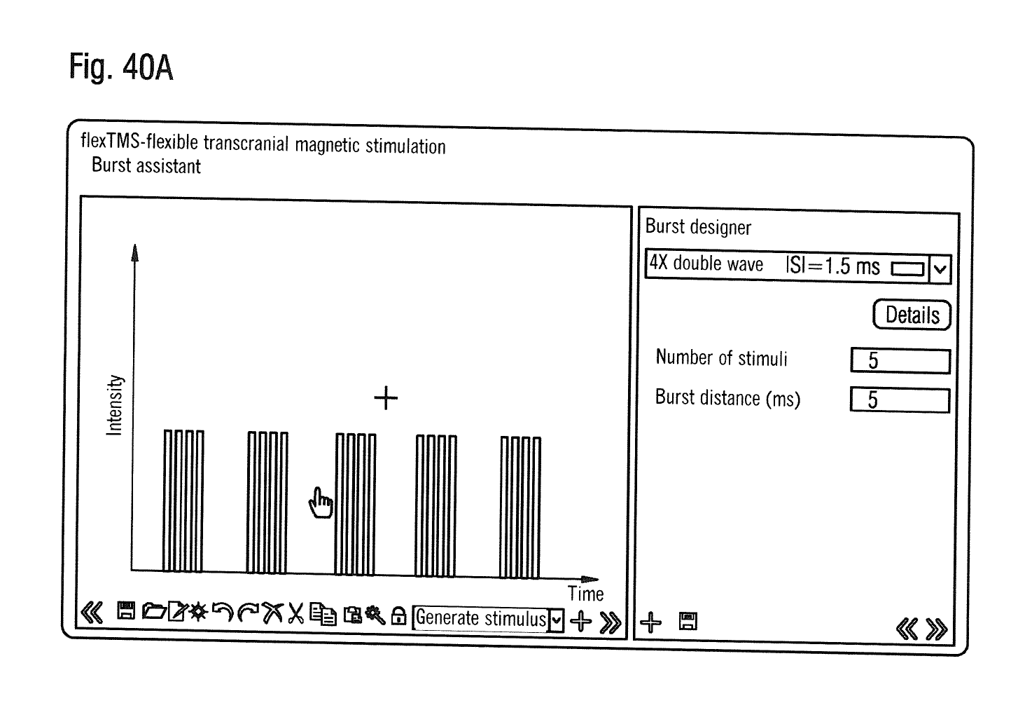

FIG. 39 is a diagram to illustrate a stimulus designer used in the user-editor;

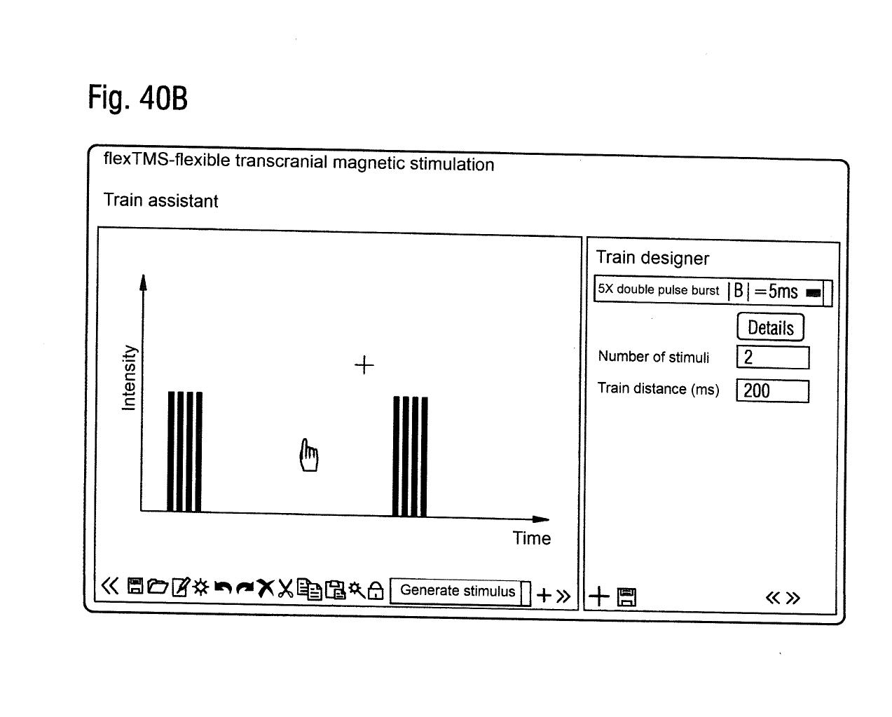

FIGS. 40A, 40B are diagrams to illustrate a pulse packet and pulse train assistant which are used in the user-editor;

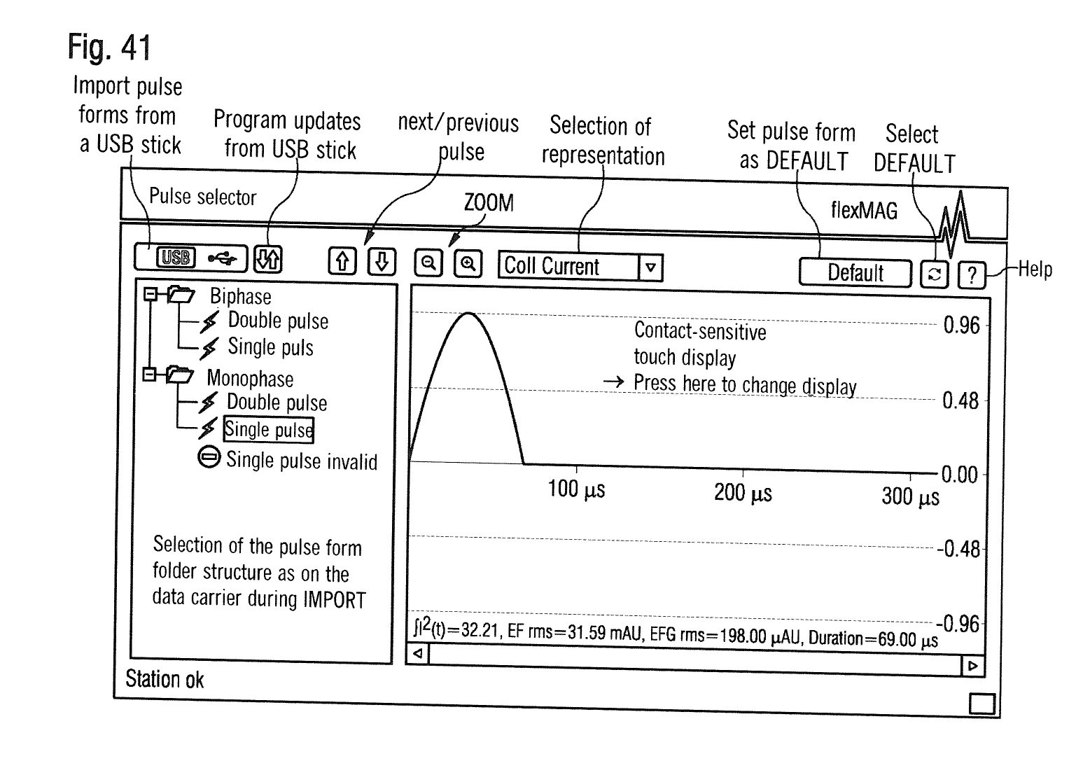

FIG. 41 is a diagram to illustrate a pulse selector used in one possible embodiment variation;

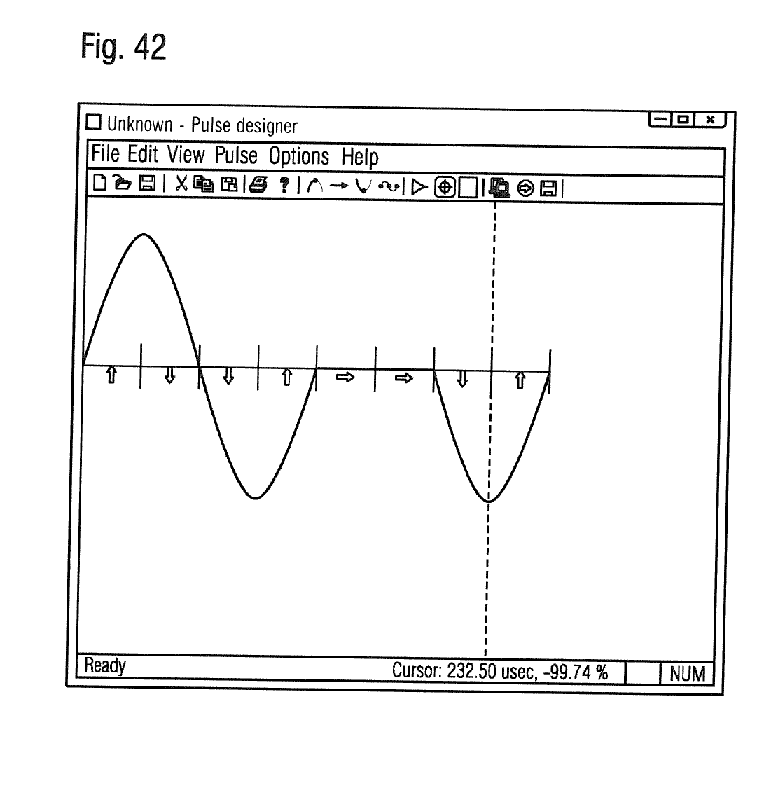

FIG. 42 shows an example of a pulse composed using a user-editor;

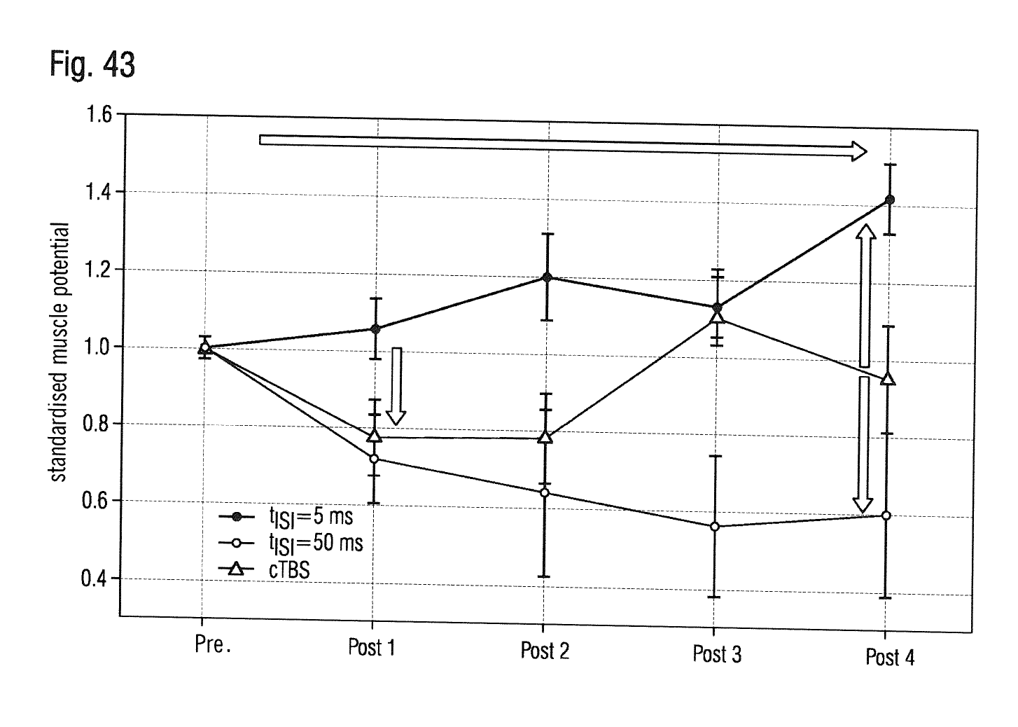

FIG. 43 is a diagram to illustrate a standardised muscle potential as can be caused by the magnetic stimulator in accordance with the invention in comparison with a conventional magnetic stimulator;

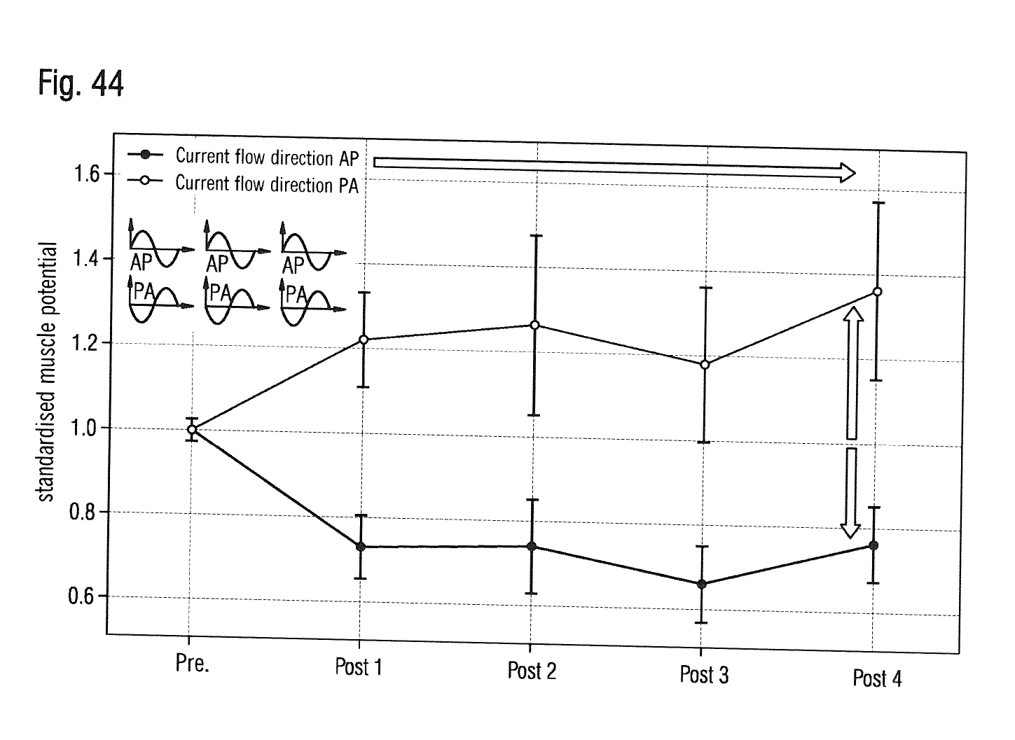

FIG. 44 is a diagram to illustrate a standardised muscle potential as can be caused by the magnetic stimulator in accordance with the invention for different current flow directions;

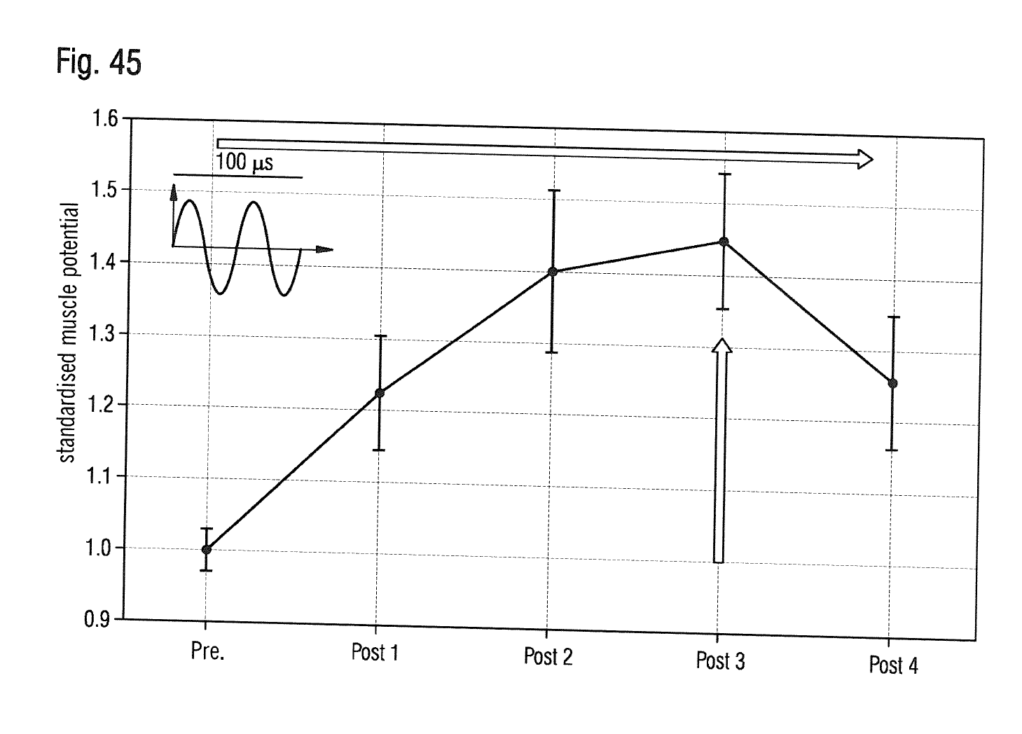

FIG. 45 is a further diagram to illustrate a standardised muscle potential as can be caused by the magnetic stimulator in accordance with the invention when using a doubled sine wave;

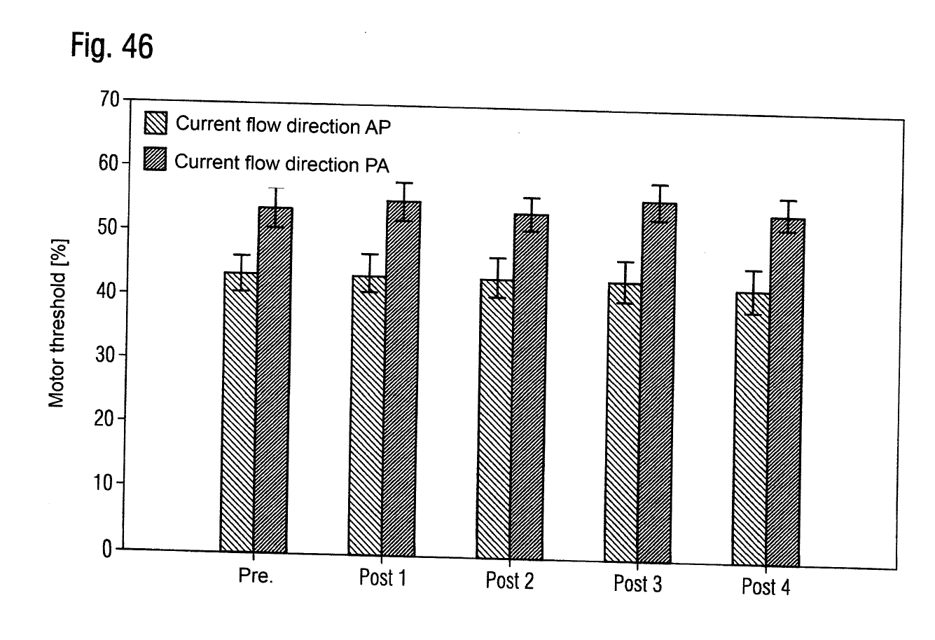

FIG. 46 shows diagrams to illustrate a motor threshold in dependence upon a current flow direction used in the magnetic stimulator in accordance with the invention.

FIG. 2 shows an exemplified embodiment of a magnetic stimulator 1 in accordance with the invention for stimulation of a tissue by a magnetic field. The tissue can be e.g. organic tissue of a patient P, especially brain tissue. In the illustrated embodiment, the magnetic stimulator 1 has a pulse generator device 2 and a programmable controller 3. The pulse generator device 2 contains at least one pulse capacitor which can be charged by a charging circuit to generate a pulse sequence, consisting of pulses, with an adjustable repeat rate. The control device 3 is a programmable control device which adjusts and/or actuates the pulse generator device Z in order to generate a complex pulse sequence PS. This complex pulse sequence can comprise individually configurable pulses. The complex pulse sequence PS generated by the pulse generator device 2 is output to a processing coil or stimulation coil 4 via a line 5. The line 5 can be a high voltage-carrying line or a high current-carrying line. The treatment or stimulation coil 4 is located in the vicinity of the tissue to be stimulated, e.g. the brain tissue of a patient P, as indicated in FIG. 2. In the exemplified embodiment illustrated in FIG. 2, the programmable control device 3 of the magnetic stimulator 1 is connected to a computer 7 via an interface 6.

A user-editor for configuration of a complex pulse sequence is preferably provided in the computer 7. The computer 7 can be a PC, a tablet computer or a laptop computer, the user-editor of which can be used to generate or configure the complex pulse sequence PS. In one possible embodiment variation, the user-editor can be displayed to a user, who is treating e.g. the patient P, via a graphical user interface, GUI. In one possible embodiment variation, the user-editor has a stimulus designer for configuration of a pulse form of individual pulses. Furthermore, the user-editor used can comprise a pulse packet assistant for configuration of at least one pulse packet consisting of pulses. Furthermore, the user-editor can also comprise a pulse train assistant for configuration of at least one pulse train consisting of pulse packets. In this way, it is possible for a user to configure and/or program a complex pulse sequence PS tailored to the individual requirements of the patient P. Thus, the complex pulse sequence PS consists of pulse trains PZ which each comprise pulse packets PP which for their part consist of a sequence of pulses. The pulse form of the pulses or individual pulses are preferably individually configurable with respect to their pulse form and/or polarity with the aid of the user-editor. In a further possible embodiment, the pulse sequence PS configured by means of the user-editor is transmitted via the interface 6 to the programmable control device 3 of the magnetic stimulator 1 and can be stored in a memory unit 8 of the magnetic stimulator 1. The memory 8 can be e.g. an EEPROM memory. The interface 6 is suitable for transmitting complex pulse patterns. For example, the interface 6 can be a USB or Ethernet interface.

In the embodiment illustrated in FIG. 2, the programmable controller 3 of the magnetic stimulator 1 is connected to a conducting electrode 10 via a separate circuit 9. The conducting electrode 10 is e.g. an adhesive electrode for conducting an EMG signal. The conducting electrode 10 is connected via a line 11 to the circuit 9 which is provided to amplify, digitise and record muscle signals. The circuit 9 can, on the one hand, output a trigger signal via a line 12 and, on the other hand, output a measurement signal via the line 13 to the programmable control device 3 of the magnetic stimulator 1. By means of the trigger signal the magnetic stimulator 1 can signal the pulse output to a recording device. The transmission of the trigger signal via the line 12 can also be effected bidirectionally. By means of the line 13 a measured signal can be returned to the magnetic stimulator 1 in order e.g. to adapt stimulation parameters of the stimulation signal output to the patient P. These stimulation parameters include e.g. the intensity or frequency of the signal. In one possible embodiment variation, the signal path 13 is deactivated. In this case, the signal path 13 is not used since a self-regulating rapid stimulation system represents a medical risk in certain cases, e.g. can cause an epileptic seizure in the patient P. In other cases, the return signal path or the return signal channel is activated in order to use the feedback for automated determination of parameters, in particular of a motor threshold. Thus, e.g. in order to determine the motor threshold approximately every 10 seconds a stimulation pulse with a specific intensity is output to the patient P and the muscle response is evaluated. With the aid of a maximum likelihood method, the intensity can be varied until a specific portion of the measured muscle responses is within a specific voltage range (e.g. 15 of 20 pulses generate muscle response potentials of >50 μV at an intensity of 65% of the maximum stimulator output). This intensity then forms the motor threshold of the respective patient P. In this embodiment variation, the determination of the motor threshold can be effected in an automated manner, whereby operational comfort for the user is increased and at the same time the determination of the motor threshold of the patient P can be effected more rapidly.

FIG. 3 shows a block circuit diagram to illustrate circuit-technology details within the magnetic stimulator 1 in accordance with the invention. In the exemplified embodiment illustrated in FIG. 3, the pulse generator device 2 contains a charging circuit 2a, an oscillation circuit 2b with a pulse switch which is connected to the stimulation or treatment electrode 4, and a coil monitoring circuit 2c likewise connected to the stimulation or treatment electrode 4. The programmable controller 3 and the different units or assemblies of the pulse generator device 2 can exchange device-internal control signals, e.g. via an internal CAN-bus.

The pulse generator device 2 contains a charging circuit 2a which is provided to recharge the pulse capacitor with an adjustable repeat rate. The pulse capacitor C_{PULS} is preferably part of an oscillation circuit in which the stimulation or treatment coil 4 is also located. The charging circuit 2a is preferably connected to a power supply network via a mains connection. The programmable control device 3 can contain a plurality of interfaces, in particular an interface 6 for connection to the computer 7 and a trigger input/output 12 for connection to the signal processing circuit 9 and an interface 13 to obtain a return signal from the conducting electrode 10. The programmable controller 3 illustrated in FIG. 3 essentially serves to control the process of the complex pulse protocols and to monitor critical parameters of the magnetic stimulator 1 and for communication with the user. In one possible embodiment variation, the programmable controller 3 has a dedicated graphical user interface, GUI, and so the programming of the complex sequence PS is possible without connection of an external computer 7.

In one possible embodiment variation, the programmable control device 3 causes the pulse generator device 2 to output the pulse sequence PS to the stimulation coil 4 only after a system check of parameters of the magnetic stimulator 1 has been successfully concluded. FIG. 4 shows a flow diagram for illustration of an embodiment variation of a system check carried out by the programmable control device 3. Thus, during the system check in one possible embodiment variation, different parameters are interrogated which relate to coil monitoring, the oscillation circuit, the charging circuit and/or a user communication. For example, with respect to coil monitoring a check is first made as to whether a treatment or stimulation coil 4 has been connected to, or plugged onto, the magnetic stimulator 1. Furthermore, monitoring is effected as to how high the coil temperature of the stimulation coil 4 is. Furthermore, it is possible to check whether all assemblies respond or react to commands of the programmable control device 3. In one possible embodiment variation, the coil monitoring circuit 2c—illustrated in FIG. 3—of the pulse generator device 2 can monitor whether a stimulation coil 4 is actually connected to the magnetic stimulator 1. In one possible embodiment, the detection of whether or not a stimulation coil 4 is provided can be effected by means of a shorting link, constructed within a coil plug, an encoding resistor or by RFID tags or by means of an impedance measurement at the stimulation coil 4. In a further possible embodiment variation, the coil monitoring circuit 2c additionally has sensors to monitor operating parameters of the stimulation coil 4. In one possible embodiment, the coil monitoring circuit 2c has temperature sensors for monitoring an operating temperature T of the treatment coil or stimulation coil 4. Thus, in particular a check is made as to whether the surface temperature of the stimulation coil 4, with which the patient P comes into contact, exceeds a temperature of e.g. 40° C. The coil monitoring circuit 2c evaluates the temperature values delivered by the temperature sensors. In one possible embodiment, the coil monitoring circuit 2c has two temperature sensors and compares the two values thereof with one another. If the two measured temperatures differ significantly from one another and if the temperature is e.g. above 40° C., by means of the programmable controller 3 a further pulse output by the pulse generator device 2 is blocked or deactivated and, if appropriate, an error is signalled to the user via a user interface.

Furthermore, the programmable control device 3 can block or deactivate the output of pulses when no stimulation coil 4 is connected to, or plugged into, the magnetic stimulator 1. In this way, e.g. the undesired formation of an arc can be prevented. In one possible embodiment variation, the monitoring of the sensors, in particular the temperature sensors, can be effected by means of at least one microprocessor. Thus, in one embodiment variation, the microprocessor can be constructed redundantly with mutual checking. Alternatively, a redundant monitoring channel can be embodied by discrete hardware.

During the system check illustrated in FIG. 4, parameters can also be checked with respect to the oscillation circuit using a pulse switch. For example, it is possible to determine how high the operating temperature at a power switch provided therein is. Furthermore, it is possible to check whether the assemblies concerned respond to commands of the programmable control device 3. Furthermore, it is possible to check e.g. whether all necessary auxiliary voltages are present.

Furthermore, the system check can check parameters of the charging circuit 2a. For example, a check is made as to whether there are voltage asymmetries at an intermediate circuit of the charging circuit 2a.

Furthermore, voltage asymmetries at the pulse capacitor C_{PULS} can be checked. Furthermore, it is possible to check whether all voltages are present in an admissible voltage range e.g. at the intermediate circuit or pulse capacitor. Furthermore, a check is made e.g. as to whether the temperature at a charging regulator of the charging circuit 2a is within a valid range.

Furthermore, the system check shown in FIG. 4 can check parameters of the user communication. For example, a check is made as to whether a user selects or has transmitted a valid pulse pattern or a valid complex pulse sequence PC.

Furthermore, a check can be made as to whether or not the user would like to cut off the current output of the pulse sequence PS. If one or a plurality of the checked pulse parameters shows that a critical state is present, or the user wishes to interrupt the pulse output, the pulse output by the pulse generator device 2 is automatically prevented or blocked by the programmable control device 3.

In one possible embodiment of the programmable controller 3, this controller has one or a plurality of microprocessors. These microprocessors can be connected to the other assemblies of the system via a real time-capable, error-tolerant or error-recognising bus, preferably a CAN-bus, and can thereby communicate with the assemblies.

In one possible embodiment, the interface to the user is formed by a standardised interface by means of specific standardised data transmission protocols, preferably USB or Ethernet. By means of this interface the programmable control device 3 of the magnetic stimulator 1 can be connected to a computer 7, e.g. a PC, laptop or tablet computer, or to a mobile terminal, in particular a smartphone or the like. Furthermore, the programmable control device 3 can be connected via corresponding interfaces to measuring and interchangeable measuring devices and can have a trigger input and a trigger output. In one possible embodiment variation, the programmable control device 3 is connected to display elements or display devices of the magnetic stimulator 1.

As shown in FIG. 3, the pulse generator device 2 of the magnetic stimulator 1 has an oscillation circuit with a pulse switch 2c. Different embodiment variations are given in this case. In one possible embodiment variation, the oscillation circuit with the pulse switch 2c is embodied with a single power switch. In a further possible embodiment, the oscillation circuit with the pulse switch 2c is constructed from a full bridge. In a further embodiment variation, the oscillation circuit with the pulse switch 2c consists of a full bridge with switched pulse capacitances.

The first embodiment variation of the oscillation circuit with the pulse switch 2c allows exclusively the outputting of biphase (sinusoidal) pulse forms/stimuli. In contrast, the embodiment variation in which the oscillation circuit with the pulse switch is constructed as a full bridge requires at least four power switches but in return offers the advantage of a largely free shape to the respective pulse form. With this embodiment variation, the complex pulse sequence can be completely parameterised by the user.

The oscillation circuit with the pulse switch 2c has at least one power switch which is connected to a driver circuit which can be controlled by the programmable control device 3. In one possible embodiment variation, this driver circuit or actuation circuit has a maximum switching frequency for the power switches. An IGBT power switch is preferably used for the power switch. The maximum switching frequency of the actuation or driver circuit is 100 kHz in one possible embodiment variation. FIG. 5 shows a block circuit diagram of a possible embodiment of a controllable driver circuit TS which is constructed for a power switch SW. The power switch is preferably an IGBT power switch. In the oscillation circuit, this IGBT power switch is located between the pulse capacitor C_{PLUS} and the stimulation coil 4, as shown in FIG. 5. In the embodiment variation shown in FIG. 5, the driver circuit TS contains a microprocessor MP which is connected via a CAN-bus to the programmable controller 3. The driver circuit TS shown in FIG. 5 has current zero crossing detection for detection of an inductance L of the treatment or stimulation coil 4. With the current zero crossing recognition, the switching behaviour of the driver can be adapted to the inductance L of the stimulation coil 4, as shown in FIGS. 6A-6E. FIGS. 6B-6E show by way of example the location in time of the current zero crossing at different inductances L and in particular in the event of a short circuit, i.e. in a short-circuited coil with residual inductance present. FIG. 6A shows the oscillation circuit connected to the charging circuit 2a, and the power switch SW contained therein. FIG. 6B shows the current zero crossing at appropriate inductance. FIG. 6C shows the progression in the case of excessively high inductance in the stimulation coil 4 and FIG. 6D shows the case when the inductance in the stimulation coil 4 is excessively low. FIG. 6E shows finally the case of a short circuit. In one possible embodiment the current zero crossing recognition in the driver circuit TS takes place via measurement of a drop in voltage via the respective power switch SW. In comparison with a current measurement at the conductor, this offers the particular advantage that the voltage measured is that also actually present on the protective component, and not a current which is provided in the conductor i.e. upstream of the IGBT module. Furthermore, in this manner of proceeding, the change in voltage takes place only when a short-term reverse recovery current, imposed by a reverse recovery effect, has also abated after the zero crossing.

As shown in FIG. 5, the microprocessor MP of the driver circuit TS can evaluate a temperature T, detected by sensors, at the oscillation circuit, in particular the stimulation coil. The driver circuit TS illustrated in FIG. 5 can contain bipolar drivers, wherein an external voltage can be returned to the microprocessor MP, as shown in FIG. 5. Asymmetrical gate actuation +18 V/12 V can be provided for safe switch-on and switch-off. Furthermore, it is possible for auxiliary voltages to be monitored by the microprocessor MP. As shown in FIG. 5, the microprocessor MP passes a pulse command to an AND-gate which can receive a redundancy signal. In one possible embodiment variation, the switch-on time is between 1 and 2 microseconds in order to reduce switch-on losses. Furthermore, in one possible embodiment variation, the switch-of time can be 8 microseconds, which, together with a discrete hardware circuit, leads to minimisation of switching over-voltages.

In one possible embodiment variation, only one single power switch SW, in particular an IGBT switch, is provided on the oscillation circuit. In this embodiment variation, the pulse form, which can be used within a complex pulse sequence, is exclusively sinusoidal. The advantage of this embodiment variation is found in the low effort required for implementation. In a preferred alternative embodiment, the oscillation circuit with the pulse switch is embodied within a full bridge. FIG. 7 shows a circuit diagram to illustrate an exemplified embodiment of a full bridge circuit for flexible pulse forms. In this embodiment, the stimulation coil 4 is connected in a full bridge with four power switches Q1, Q2, Q3, Q4 to generate pulses, the pulse form of which can be composed of pulse segments. The voltage at the pulse capacitor C_{PULS} has the pulse [and] is determined by the charging circuit 2a. The different power switches Q1 to Q4 can be actuated via an associated IGBT driver. The capacitors C1, C2 provided in the circuit as shown in FIG. 7 serve for voltage symmetrisation. Furthermore, the full bridge circuit illustrated in FIG. 7 can contain a so-called snubber circuit

SN which is provided to lower voltage peaks which can occur when an inductance L is switched off. The pulse capacitor C_{PULS} serves to store energy. The snubber circuit SN contains some capacitors C3 to C10 which are connected via resistors R1, R2 to the stimulation coil 4. The snubber capacitors have e.g. a capacitance between 100 to 300 nF. The snubber resistors R1, R2 can have e.g. a resistance value of 1 to 10 Ohm. In parallel with the IGBT power switches Q1 to Q4, free-wheeling diodes D1 to D4 can be provided in each case, as shown in FIG. 7. In one possible embodiment, the symmetrisation capacitors C1, C2 can each have a capacitance of 0.1 to 1 microfarad. The pulse capacitor C_{PULS} preferably has a relatively high storage capacity of more than 20 μF, e.g. 66 μF. The capacitance of the pulse capacitor C_{PULS} can amount to some mF.

FIG. 8 shows diagrams for illustration of a current flow in the full bridge circuit shown in FIG. 7. Since the current flow comes about through the LC oscillation circuit, which contains the pulse capacitor C_{PULS} and the stimulation coil 4, the current flow has a sinusoidal progression. The amplitude of the oscillation is determined by the charging voltage of the pulse capacitor C_{PULS}. The frequency of the oscillation results from the capacitance C_{PLUS} of the capacitor and the inductance L of the coil 4. As a segment of a sinusoidal oscillation, holding phases can also be created with the full bridge circuit illustrated in FIG. 7, i.e. almost any number of different pulse forms can be created. For this purpose, the coil 4 is short-circuited in phases during conduction of current, as shown in FIG. 8A. At this juncture, the energy remains within the coil 4. Thus, damping is taken into account, this being able to occur both during the sinusoidal oscillations and also during the holding phases. The damping is brought about by the ohmic losses of the stimulation coil 4 of the pulse capacitor C_{PLUS} and the electric lines. Furthermore, the current progression is damped by time losses at the power switches Q. In the embodiment variation illustrated in FIG. 7, the power switches Qᵢ are embodied by IGBTs which each have free-wheeling diodes D1-D4. Thus, in the embodiment variation of the full bridge circuit illustrated in FIG. 7, it is sufficient during the holding phases to keep only one power switch Qᵢ closed. Thus e.g. for the holding phases at a positive level only the power switch Q1 must be closed, wherein the diode D4 on the power switch Q4 automatically closes the switch Q4 for the required current direction.

When the full bridge circuit illustrated in FIG. 7 is used, three possible segment types result, with which a single pulse can be constructed or configured, namely a rising portion (sinusoidal with a time constant T=L×C_{PULS}), a constant portion and a falling portion (sinusoidal with a time constant T=L×C_{PULS}, wherein ohmic losses are disregarded.

These three segments can be strung together in almost any lengths and in any combinations. Any pulse forms can thereby be generated within broad limits. Thus, switching losses and a minimum switch-on duration are taken into account since the power switches Q cannot be switched at a random frequency.

FIG. 8A shows different current flow phases through the full bridge circuit illustrated in FIG. 7. FIG. 8B shows associated segments for a generated single pulse.

By way of example, pulse forms with a representation of the associated switch positions are illustrated in FIGS. 9, 10 and 11. Thus, FIG. 9 shows the actuation of the full bridge circuit with changing polarities. FIG. 10 shows the actuation of the full bridge circuit with a single polarity. FIG. 11 shows the actuation of the full bridge circuit with holding phases.

FIG. 12 shows an extension of the full bridge circuit to at least two pulse capacitors. For this purpose, a plurality of charging circuits can be provided. An advantage in the full bridge circuit illustrated in FIG. 12 is that the different pulse capacitors can be charged to different voltage levels. In this way a still higher repeat rate than 1 kHz is possible. The higher repeat rates can be achieved in that the required pulse energy is provided in an alternating manner from the different pulse capacitances. A further advantage of the embodiment variation consists of possible use of different time constants which, in contrast to the simple full bridge circuit shown in FIG. 7, opens up the possibility of configuring or forming strongly asymmetric pulse forms, as shown in FIG. 13. The use of asymmetric pulse forms within the complex pulse sequence PS potentially allows the stimulation of further areas of the brain in the patient P being treated. FIG. 13 shows by way of example a strongly asymmetric pulse form with two time constants T₁ and T₂.

The pulse generator device 2 used in the magnetic stimulator 1 contains a charging circuit 2a which is provided to recharge the pulse capacitor C_{PLUS} with a high adjustable repeat rate. In one possible embodiment, the recharging of the energy, lost during the pulse output, of the pulse capacitor C_{PULS} takes place, e.g. within a time of 1 ms. In this embodiment variation, the maximum repeat rate is 1 kHz. In one possible embodiment the charging current for charging the pulse capacitance is about 100 A.

In one possible embodiment, the charging circuit 2a used in the pulse generator device 2 is a linear charging circuit. In a further alternative embodiment, the charging circuit used in the pulse generator device 2 is a clocked charging circuit.

FIG. 14 shows a block circuit diagram for a possible embodiment of a linear charging circuit 2a, as can be used inside a pulse generator 2 of the magnetic stimulator 1. The charging circuit 2a serves to charge the pulse capacitor to a specific voltage level U_{VOLL} and to recharge the energy lost after pulse output within the short time of e.g. a maximum of 1 ms. The linear charging circuit 2a illustrated in FIG. 14 has a mains adapter NT for connection to a power supply network, an intermediate energy circuit EZK for intermediate storage of the electrical energy supplied by the mains adapter NT, and a charge regulator which is connected to the oscillation circuit of the pulse generator device 2. The mains adapter NT used can be a standard mains adapter or a transformer with a rectifier. The starting voltage U_{PS} of the mains adapter NT can be e.g. of an order of magnitude of 2000 to 4000 V. The mains adapter NT illustrated in FIG. 4 can be designed in different embodiment variations either as a single-phase or a three-phase mains adapter NT. By reason of the favourable duty cycle in the pulse output, conventional single-phase mains adapters are preferably used in order to provide the required pulse power.

For the linear charging circuit 2a in accordance with FIG. 14, an intermediate energy circuit EZK is provided on the DC side of the mains adapter. This intermediate energy circuit EZK serves for buffering and intermediate storage of the electrical energy supplied by the mains adapter NT. The intermediate circuit voltage in the intermediate energy circuit EZK is thus preferably selected to be greater than a maximum desired voltage U_{SOLLmax} at the pulse capacitor C_{PULS} of the oscillation circuit, in order to exploit the steepness of an RC charging curve as shown in FIG. 15 and therefore to permit a rapid energy recharge in the intermediate energy circuit EZK. A capacitor provided in the intermediate energy circuit EZK has a capacitance C_{ZW} which is preferably substantially greater than the pulse capacitance C_{PULS} of the pulse capacitor and so the largest possible energy store can be provided.

The linear charging circuit 2a illustrated in FIG. 14 contains a charging regulator LR which is connected to the intermediate energy circuit EZK. The charging regulator LR charges the pulse capacitance of the pulse capacitor to a desired value voltage U_{SOLL}. For this purpose, the charging switches S1 to S4 of the charging regulator LR are actuated in dependence upon the actual voltage U_{C} provided at the pulse capacitor. The charging switches S1 to S4 can preferably be formed as IGBT switches by reason of the high voltage and rapid switching phases. The actual voltage at the pulse capacitor is detected and processed by a microprocessor MP of the charging regulator LR. The microprocessor MP of the charging regulator LR then controls the charging switches S1 to S4. In addition, the temperatures at the charging and discharging resistors R1 to R4 can be monitored by the microprocessor MP. The switch S5 in combination with the resistor R5 is provided for an emergency discharge of the pulse capacitor in the event of a problem. The switch S5 is thus preferably formed as a high voltage relay. This high voltage relay can be switched via the microprocessor MP. In one possible embodiment variation, the high voltage relay can be switched by reason of the redundancy by a discrete hardware circuit (not shown).

The microprocessor MP of the charging regulator LR within the linear charging circuit 2a can be connected, in one possible embodiment, via a CAN-bus to the device controller or programmable control device 3. In one possible embodiment, the microprocessor MP is used as a redundant component. In this embodiment variation, two microprocessors are constructed which can be connected in the same manner. These two microprocessors mutually check their measurement and actuation results. If, e.g., one of the two microprocessors fails or if the two microprocessors output contradictory results, in one possible embodiment variation, an emergency discharge can take place with the aid of the switch S5 and of the resistor R5. If in an alternative embodiment variation, no redundant microprocessors are constructed, then a further redundancy circuit is preferably embodied in order to monitor the voltage. If an error occurs, in particular if an overvoltage occurs, this checking circuit or checking entity then switches the high voltage off with the aid of the switch S5 and the resistor R5. This redundancy circuit is provided in particular when the magnetic stimulator 1 is used as a medical device.

FIG. 16 shows signal diagrams for illustration of the behaviour of the charging switches S1 to S4 within the charging regulator LR of the linear charging circuit 2a, as illustrated in FIG. 14. In the embodiment variation illustrated in FIG. 16, the actuation of the charging switches S1 to S4 is effected via bipolar driver stages directly by a microprocessor MP of the charging regulator LR. FIG. 16 shows the voltage progression U_{C} at the pulse capacitor and necessary actuation signals for the charging switches S1 to S4 for different scenarios.

The charging circuit 2a used within the pulse generator device 2 of the magnetic stimulator 1 for recharging the pulse capacitor with an adjustable repeat rate can be a clocked charging circuit in a further embodiment. FIG. 17 shows a block circuit diagram to illustrate an exemplified embodiment of a clocked charging circuit 2a. The clocked charging circuit 2a has a mains adapter NT for connection to a power supply network, a first DC/DC switching regulator for continuous operation, an intermediate energy circuit EZK for intermediate storage of the electrical energy supplied by the first DC/DC switching regulator, and a second DC/DC switching regulator for pulsed operation, which is connected to the current circuit of the pulse generator device 2, as shown in FIG. 17. The mains adapter comprises a diode full bridge and an input filter. The first DC/DC switching regulator connected to the mains adapter is arranged for continuous operation, e.g. for a 2000 W continuous rating. The first DC/DC switching regulator continuously charges an intermediate circuit capacitor C_{S} of an intermediate energy circuit EZK at a preset voltage, e.g. 400 V. The intermediate energy circuit EZK is preferably arranged such that the stored energy in the intermediate circuit capacitor is large compared with the maximum storable energy in the pulse capacitor C_{PULS} of the oscillation circuit. The second DC/DC switching regulator of the clocked charging circuit 2a illustrated in FIG. 17 is arranged for pulsed operation for the transmission of large quantities of energy, e.g. of up to 5000 W. Thus, the duty cycle is preferably suitably dimensioned. The second DC/DC switching regulator charges the pulse capacitor C_{PULS} during pauses in the stimulation. The second DC/DC switching regulator is not actuated when the oscillation circuit SW is closed, as illustrated in FIG. 17, and a pulse is output. The second DC/DC switching regulator acts directly on the pulse capacitor C_{PULS} of the oscillation circuit and must therefore exclusively drive a capacitive load. This leads to high ripple contents by reason of the clocked charging process being of no consequence since the charging voltage at the pulse capacitor C_{PULS} is used for pulse output only when the second DC/DC switching regulator is no longer active.

In one possible embodiment, a power form correction, PFC, takes place at the first DC/DC switching regulator of the clocked charging circuit 2a. This switching step serves to carry out a normatively prescribed power form correction from a specific nominal power. With such a power form correction, it is possible to ensure that the current draw from the power supply network is as sinusoidal as possible. FIG. 18 shows a possible current flow at the converter input in comparison with a purely sinusoidal current draw. The mode of operation of the power form correction consists of controlling the drawn current in dependence upon the sinusoidal voltage measured at the input (type of operation CCM=continuous conduction mode). The continuous sinusoidal line shown in FIG. 18 therefore indicates an ideal condition. The other, broken line represents the current draw with the PFC and shows switching times of the converter (it represents an approximation to the ideal condition).

One possible embodiment of a power form correction (PFC) circuit as a boost converter is illustrated in FIG. 19. If the switch S1 provided is closed, a coil current is built up by the coil L. If the switch is then opened, the current flows via the diode D into the intermediate circuit capacitor C_{S}, wherein the coil current falls. Upon reaching a lower threshold value, the switch S1 is closed and the coil current rises. The embodiment variation shown in FIG. 19 has the particular advantage that, by reason of the low voltages at the intermediate circuit capacitor (e.g. 400 V) the switch S1 can also be formed as a MOSFET. Alternatively, the switch S1 can also be embodied as an IGBT power switch.

Different embodiment variations are possible for the charging regulator within the clocked charging circuit 2a illustrated in FIG. 17. In one possible embodiment variation, the second DC/DC switching regulator is embodied as a push-pull flux converter as shown in FIG. 20. In this embodiment variation, the pulse capacitor C_{PULS} can only be charged. Discharging of the pulse capacitor is effected via a further switch and a discharge resistor in a similar manner to the case of the linear charging circuit. In this embodiment variation the pulse capacitor C_{PULS} can thus be charged only with one polarity and a reversal of polarity is not readily possible. FIG. 21 shows a current flow through the pulse capacitor C_{PULS}. In the illustrated embodiment variation, the current flow I is uninterrupted, i.e. there is a continuously flowing charging current. By virtue of the actuation of the transformer with an H-bridge, this transformer is alternately loaded in both current directions.

In a further embodiment variation, the charging regulator LR of the clocked charging circuit 2a illustrated in FIG. 17 can be formed as a flyback converter for charging the pulse capacitor C_{PULS}. FIG. 22 shows a circuit diagram of an embodiment variation in which the charging regulator LR is formed as a flyback converter. In this way, the switching effort is reduced compared with the push-pull flux converter illustrated in FIG. 20. In the variation of the charging regulator LR illustrated in FIG. 22, the pulse capacitor C_{PULS} is only charged when energy is drawn from the transmission transformer i.e. the charging current is interrupted as shown in FIG. 23. If the switch S1 of the charging regulator LR illustrated in FIG. 2 is closed, the current increases through the transformer, wherein energy is transported. In contrast, if the switch S1 is open, energy flows from the transformer into the pulse capacitor, wherein the current flow in the transformer falls until the switch S1 is closed. A disadvantage of this is the intermittent operation of the charging current, wherein when the same amount of energy is transmitted, a higher current maximum than in the push-pull flux converter is required, as shown in FIG. 20. It is also disadvantageous with the charging regulator LR illustrated in FIG. 22 that the pulse capacitor C_{PULS} can be charged with only one polarity, i.e. a reversal of polarity is not readily possible.

In a further embodiment variation of the charging regulator within the clocked charging circuit illustrated in FIG. 17, this charging regulator is formed as a flyback converter for charging and discharging the pulse capacitor. In this embodiment variation the flyback converter is expanded by a further switch, as shown in FIG. 24. In this way, the circuit topology can be used both to charge and also to discharge the pulse capacitor C_{PULS}.

In the above-illustrated embodiment variations of the charging regulator LR, in each case the measurement devices for the voltages produced and the associated microprocessor for actuation of the switches are not shown for the sake of clarity.

Provided that the switch S7 in the embodiment variation illustrated in FIG. 24 is kept open and the switch S1 is clocked, the converter behaves like the previously described embodiment variation according to FIG. 22. In contrast, if the switch S1 is kept open and the switch S7 is actuated in a clocked manner, energy is first transmitted from the pulse capacitor C_{PULS} into the transformer (switch S7 closed) and then from the transformer to the intermediate circuit capacitor C_{PULS} (switch S7 closed). In this embodiment, the effects of the voltage level of the intermediate circuit capacitor C_{S} and therefore also of the first DC/DC switching regulator are to be taken into consideration. For example, the first DC/DC switching regulator attempts to keep the voltage at the intermediate circuit capacitor C_{S} at a voltage of 400 V, wherein, however, it can be tolerant up to 500 V charging voltage. This voltage can be achieved when the intermediate circuit capacitor C_{S} has a voltage level of 400 V and additionally the pulse capacitor is completely discharged with respect to the intermediate circuit capacitor C_{S}. In the embodiment variation illustrated in FIG. 24, the switch S7 cannot be formed as a MOSFET by reason of the relatively high voltage level. Thus, the switch S7 is preferably designed in this embodiment variation as an IGBT switch. An advantage of the circuit topology illustrated in FIG. 24 consists of achieving a recovery of energy through the active discharging process.

The charging circuit 2a of the pulse generator device 2 within the magnetic stimulator 1 can be formed as a linear charging circuit or as a clocked charging circuit. For example, FIG. 14 shows an embodiment with a linear charging circuit. In contrast, FIG. 17 shows an embodiment variation with a clocked charging circuit. Compared with the clocked charging circuit, the linear charging circuit requires a large intermediate circuit capacitor suitable for high voltage and having a capacitor voltage of e.g. over 2000 V. The resistors, via which the energy from the intermediate circuit is transmitted into the pulse capacitor C_{PULS}, lead, in addition to the pulse losses during the pulse output to the stimulation coil 4, to additional losses, wherein this can be associated with a significant rise in temperature. In contrast, a clocked charging circuit stores the intermediate circuit energy at a relatively low voltage level of e.g. 400 V. The required high voltage for pulse output occurs only at the pulse capacitor itself or only at an output of the switched mode mains power supply. The losses within the clocked charging circuit are thus lower than when using a linear charging circuit. For this reason, the clocked charging circuit can be constructed substantially more compactly than the linear charging circuit.

In addition, the clocked charging circuit, which is illustrated by way of example in FIG. 17, has a higher degree of effectiveness than the linear charging circuit. Thus, in a preferred embodiment of the magnetic stimulator 1 in accordance with the invention, a clocked charging circuit is used as a charging circuit 2a of the pulse generator device 2.

In a preferred embodiment of the magnetic stimulator 1 in accordance with the invention, the programmable control device 3 of the magnetic stimulator 1 can be connected to a computer 7 via an interface 6, on which computer a user-editor is provided to configure the pulse sequence PS. This user-editor is preferably a graphical editor which can be executed e.g. by the computer and can be displayed to the user via a graphical user interface (GUI) of the computer. The user is e.g. a user who is treating the patient P. In a further possible embodiment, the user-editor is performed on a computer (embedded PC) installed within the magnetic stimulator 1. In this embodiment variation, the magnetic stimulator 1 has a suitable graphical user interface (GUI).

FIG. 25 shows by way of example an operating process for configuration or parameterisation of a pulse with a particular pulse form. In one possible embodiment, the pulse form is first created with the aid of a particular pulse designer application. This created pulse can then be exported into a stimulator format. Then it is transmitted directly to the magnetic stimulator 1 via an interface. The pulse can be further processed in order to carry out an experiment and/or a session or procedure. For this purpose, the pulse can be charged with a pulse intensity application. It is thereby possible to adjust the desired pulse intensity or to generate a series of pulses. Furthermore, it is possible for the order of the pulses to be randomised via a particular randomiser application for the respective procedure. After creating the pulses, these pulses can be loaded e.g. onto a USB stick and can be copied into the magnetic stimulator 1 via a USB interface. The created pulses can also be copied into the magnetic stimulator 1 via another communication method. In one possible embodiment variation, the pulse created in this way with the particular pulse form and/or pulse polarity can be stored for further use within a memory of the magnetic stimulator 1.

FIG. 26 shows a diagram to illustrate a stimulus or pulse which consists of a single wave (FIG. 26A) or of a double wave (FIG. 26b). The illustrated stimulus consists of a single, a double or a multiple sinusoidal oscillation of the current through the stimulation coil 4. The stimulation pulse has an intensity I₀ and can be triggered at a defined time t by the user or corresponding to the formed complex pulse protocol. The polarity of the stimulus or pulse can preferably be changed, i.e. the first sinusoidal oscillation is reflected around the time axis. FIG. 26 shows the illustration of a stimulus or pulse for a positive single and double oscillation. The stimulus can be symbolised hereinunder by a rectangle as indicated in FIG. 26.

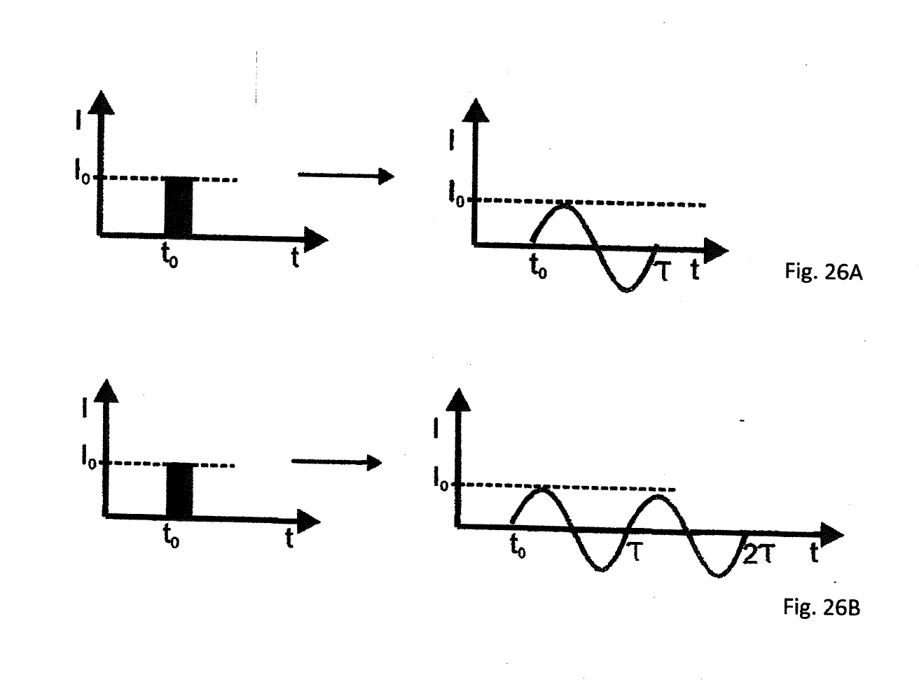

FIG. 27 shows double pulses (paired pulses). Two directly successive stimuli or pulses with the same or different amplitudes are designated as double pulses.

FIG. 27 shows the schematic illustration of a double pulse with the associated current time progression through the stimulation coil 4. The time interval between the two stimuli or pulses is designated by t_{PP} and the difference in intensity by ΔI. FIG. 27 shows the two double pulse variations most frequently used, within a complex pulse sequence PS. In one possible embodiment variation, an evaluation pulse EP is formed by such a double pulse.

The time interval t_{ISI} between stimuli with the same intensity I is designated as an interstimulus interval. The pulse sequence or the pulse protocol PS constitutes a serial arrangement in time of different stimuli or pulses, packets/bursts and double pulses with a defined property, which is automatically processed and/or output. The pulse form or stimulus form is the curve shape of the current time profile through the stimulation or treatment coil. In the case of biphase stimulation of the patient P, these are e.g. single, double and multiple waves.

FIG. 29 shows the structure of a pulse packet PP within a pulse train PZ of a complex pulse sequence PS. A pulse packet or pulse burst PP designates a container of n stimuli or pulses with an interstimulus interval t_{ISI}. Within a pulse packet or pulse burst PP, the intensity I, the polarity and the interstimulus interval of all pulses or stimuli are kept the same. A special case with n=4 stimuli is designated as a quadro-pulse stimulation.

FIG. 30 shows a diagram to clarify a packet interval or interburst interval. The packet interval or interburst interval t_{IBI} is the time interval between two pulse packets or pulse bursts. The two successive pulse packets PP are not always identical.

FIG. 31 shows a diagram to illustrate a single wave. The single wave constitutes the simplest stimulus form or pulse form of the biphase stimulation. The single wave consists of precisely one single sinusoidal oscillation with a preset period duration τ, as illustrated in FIG. 31.

FIG. 32 shows a diagram showing a double wave. The double wave consists of two sinusoidal full oscillations, as shown in FIG. 32. It is thus possible to have any number of sinusoidal oscillations in succession. By reason of the system-imposed device damping, however, the amplitude is diminished exponentially, whereby practical use is made of more than two oscillations only rarely.

FIG. 33 shows by way of example a complex pulse sequence PS having a plurality of pulse trains PZ which each consist of pulse packets PP which for their part consist of a sequence of pulses. The pulse train PZ designates a container of n different pulse packets or pulse bursts PP and forms an uppermost nesting level of a complex pulse sequence PS or of a complex pulse protocol, as shown in FIG. 33. Various different pulse trains PZ can be provided in succession. The time interval between two pulse trains PZ is designated as an intertrain interval τ_{ITI}.

The repeat rate indicates the number of stimuli or pulses per unit of time. While conventional stimulators usually achieve a repeat rate of up to 100 Hz, with the pulse generator device 2 of the magnetic stimulator 1 in accordance with the invention it is possible to set a repeat rate of up to 1 kHz and higher.

A basic protocol of a complex pulse sequence PS consists of pulse packets PP and the individual pulses or stimuli contained therein. The parameterisation of a basic protocol can indicate e.g. the interstimulus interval t_{ISI} or the pulse form or the proportion of pulses per pulse packet PP and the packet interval τ_{IBI}.

FIG. 34 shows a protocol variation or a complex pulse sequence with an evaluation pulse EP contained therein. This evaluation pulse EP is provided between two pulse packets PP and can be formed e.g. as a double pulse. In general, a trigger signal is triggered from the magnetic stimulator with respect to this evaluation pulse EP in order thereby e.g. to start an EMG amplifier in order to measure a motor muscle response. In addition to the option of parameterisation of a basic protocol, the following parameters can be adjusted in the protocol variation illustrated in FIG. 34: namely the pulse intensity of the evaluation pulse (0 to 100%), a pulse intensity difference AI between the two pulses of the double pulse which forms the evaluation pulse EP (e.g. ΔI=+/−20%), an interval from the last pulse packet t_{EV} (e.g. 100 ms) and an interval to the next pulse packet t_{DELAY} (e.g. likewise at least 100 ms).

In one possible embodiment or protocol variation, the polarity of the individual pulses or stimuli between the different pulse packets PP of the complex pulse sequence PS can be reversed. If, e.g. the pulses of the first pulse packet PP are positive pulses, the polarities of the pulses within the subsequent pulse packet PP can be negative. A reversal of polarity of the pulses within a pulse packet PP is not generally provided.

In one possible embodiment variation of the magnetic stimulator 1 in accordance with the invention, an I-wave latency time is determined. The I-wave latency time is different from one individual to another or one patient to another and can be within a range of 1 ms to 2 ms for the fundamental wave. All further I-wave latencies are integer multiples of this fundamental latency time. In one possible embodiment, the I-wave latency time of the patient P is determined by the output of double pulses (pulse-stimulation pairing) with different interstimulus intervals by measurement of a motor muscle response. Thus, the interstimulus interval is continuously adjusted until a maximum motor muscle response is measured. This interstimulus interval corresponds to the I-wave latency time of the patient.

During application of complex pulse protocols or pulse sequences PS, as required to induce a change in plasticity in a human brain, an adaptation of protocol parameters to the determined I-wave latency time during treatment produces a maximum effect.

FIG. 35 shows by way of example an operating sequence such as can take place with the magnetic stimulator 1 in accordance with the invention. The treatment of a patient P or the exposure of tissue to a magnetic field takes place within a so-called procedure (session). During the procedure, a complex pulse sequence is output via the stimulation coil 4 to the tissue to be investigated. The complex pulse sequence PS consists in the simplest case of individual pulses or stimuli. Complex pulse sequences PS, which are output during the procedure, consist of pulse trains PZ. Pulse trains PZ consist for their part of pulse bursts of pulse packets PP. The pulse bursts or pulse packets PP contain stimuli or pulses. A stimulus can be a single pulse but also, as shown in FIG. 35, a multiple double pulse. With the magnetic stimulator 1 in accordance with the invention, it is possible for a user to configure a complex pulse sequence PS individually. In one possible embodiment, after configuration of a pulse with respect to its pulse form or after configuration of a complex pulse sequence, the editor checks whether the configured pulses or the configured pulse sequence PS is admissible.

FIG. 36 shows a display on a graphical user interface, GUI, for clarification of the mode of operation of a user-editor which can be used in the magnetic stimulator 1 in accordance with the invention. In FIG. 36 a pulse frequency is formed during a session or a procedure and consists of nine individual pulses with a biphase wave form. As shown in FIG. 36, the intensity I can be selected in different variations. Furthermore, it is possible for the user to set trigger times. At each set trigger point, the magnetic stimulator 1 outputs a signal via an interface, which signal can be used by a recording device to store the muscle response following this stimulus.

In the user-editor, the pulse trains PZ and the pulse bursts or pulse packets PP can each be produced by a dedicated assistant. Thus e.g. in a dropdown box a “Burst” for a pulse packet or “Train” for a pulse train can be selected by the user. Pulse trains PZ are based on pulse packets and pulse packets PP are based on stimuli. A session or procedure can be stored as a stimulus or pulse sequence PS and can be used further in a burst designer of the user-editor. In one possible embodiment, the user-editor contains a stimulus designer, a pulse packet assistant PPA and a pulse train assistant PZA. These assistants are particularly suitable when large intervals occur between individual pulses.

FIG. 37 shows a diagram in which a burst PP is added by the user in the displayed user-editor.

FIG. 38 shows a diagram in which a pulse train PZ is added by the user via the user interface.

FIG. 39 shows by way of example a stimulus designer for configuration of a stimulus or pulse. The user has the option of changing features of the stimuli or pulses e.g. by clicking on “Detail”. For example, the user can adjust the starting polarity or the period duration of the stimulus or pulse or the respective wave. In one possible embodiment, each formed or configured stimulus or pulse can be stored and be downloaded from this memory for further processing. The pulse sequences PS can be evaluated with respect to their effects on the patient P and/or, with respect to their pulse structures, they can be correlated with measurement results and/or effects on the patient.

FIGS. 40A, 40B show by way of example, a pulse packet PP formed with a burst assistant PPA. FIG. 40B shows a pulse train PZ formed with a train assistant PZA.

In a further possible embodiment, it is possible with the aid of the pulse selector, as shown by way of example in FIG. 41, to configure a pulse form of a pulse. In the exemplified embodiment illustrated in FIG. 41, a selection screen is formed in two parts. The pulse selector preferably runs directly on the device and serves for selection of the protocols stored on the device. In this way, operation of the magnetic stimulator 1 is also possible without connection of an external PC. In a left-hand region, a pulse form can be selected, which is graphically illustrated in the right-hand part of the selection screen. Valid and invalid pulses can be characterised accordingly in the selection tree in the left-hand region. The characteristic times of the different pulses can also be illustrated. The type of curve, i.e. coil current, electrical field or electrical field gradient, can be selected by a dropdown field in the menu.

With the aid of a pulse designer, it is possible to compose or establish a pulse form of the respective pulse.

FIG. 42 shows e.g. a composed pulse which consists of a sinusoidal wave, two pauses and a negative half wave. By double-clicking, it is possible to edit the time duration of the individual portions. The length of the different portions of the pulse can also be changed by dragging with a mouse.

The magnetic stimulator 1 in accordance with the invention can be used for magnetic stimulation of organic tissue. The magnetic stimulation is a non-invasive, almost pain-free method with which nerves in the tissue are influenced by a magnetic field, which can vary over time, through induction in the electrical activity thereof. Thus, the nerves can be activated or inhibited.

The stimulation coil 4 of the magnetic stimulator 1 is placed close to a surface of the skin. The stimulation coil 4 generates a magnetic field which can vary rapidly over time and which penetrates the tissue. This penetrating magnetic field brings about induction into electrically conductive regions of the tissue. In further applications, it is also possible to introduce the stimulation coil 4 into the tissue.

The use of the magnetic stimulator 1 in accordance with the invention requires no special preparation whatsoever of the skin surface of the patient P. The magnetic stimulator 1 can generate a magnetic field which passes through clothing, hair etc. and produces a stimulation. Even deep areas can be reached since the magnetic field penetrates bone structures such as e.g. the calvarium. The depth of penetration is limited to several centimetres.

Successful stimulation depends on the strength and orientation of the electric field induced by the stimulation coil 4 and the pulse form set on the magnetic stimulator 1. The stimulation thresholds determined each apply to an investigation procedure or session since they depend greatly on the physiological make-up of the respective patient (tiredness, nervousness or e.g. blood-sugar level).

In order to make the magnetic stimulation between different patients or subjects comparable, the stimulation intensity is preferably normalised with respect to the individual motor stimulation threshold. The motor threshold is defined as the minimum stimulation strength which is sufficient to generate a certain muscle action potential in a relaxed muscle in at least half of cases. The threshold obtained in the relaxed muscle is for this reason designated as a resting motor threshold, RMT. The active motor threshold AMT can in the same way be determined in the pretensioned muscle and is usually 5 to 20% below the resting motor threshold RMT. The magnetic stimulator 1 in accordance with the invention permits the output of different pulse forms which can be self-composed. In one possible embodiment, the intensity I of the stimulation pulses to be output can be adjusted by means of a setting wheel on a user interface of the magnetic stimulator 1. Furthermore, in one possible embodiment a stored pulse form can be selected by means of a selection switch via a pulse selector on a display.

By using a further setting wheel, it is possible in one possible embodiment to adjust the repeat frequency or the repeat rate. In a further embodiment, it is possible to use a further setting wheel to select the pulse sequence duration i.e. the maximum length of a pulse sequence is output.

In a single pulse operation of the magnetic stimulator 1, upon actuation of a button, a single stimulation pulse with the selected pulse form is output. In a repeat operation of the magnetic stimulator 1 a pulse sequence with a set repeat frequency or repeat rate is output as long as a specific button is kept pressed.

In possible embodiments, the currently set values for the pulse intensity, pulse sequence, pulse sequence duration and pulse form can be stored by the user pressing a memory button. The stored values are also obtained when the magnetic stimulator 1 is switched off. It is thereby possible e.g. after switching on the magnetic stimulator 1 to retrieve a previously stored set of standard settings quickly and easily.

In one possible embodiment, the magnetic stimulator 1 changes to a standby mode provided no operating element has been actuated for a preset time. In order to terminate the standby operating mode, any operating element e.g. on a front plate of the magnetic stimulator 1 can be actuated. In this way, the magnetic stimulator 1 is placed in an operationally ready state and a corresponding display lights up.

In order to trigger single pulses, the magnetic stimulator 1 is switched on, wherein a check is made as to whether a stimulation coil 4 is connected. Thus, the desired pulse intensity can then be selected on the setting element. Furthermore, a pulse frequency is set. By actuation of a particular operating element, e.g. a pneumatic foot switch, the stimulation coil 4 can be set or activated with precision. By actuation of a pulse button, a single pulse is then output.

In order to interpret a pulse sequence, in particular a complex pulse sequence PS, it is possible to change e.g. to a long-term display mode, wherein a desired pulse sequence duration is selected. After actuation of a pneumatic foot switch for activation of the stimulation coil 4, a pulse button can be actuated and so the desired pulse sequence is output to the patient P for as long as the respective button is kept pressed. After the set pulse sequence duration has been reached, the pulse output is stopped automatically even if the button remains pressed.

With the magnetic stimulator 1 in accordance with the invention, it is possible to generate stimuli or pulses with an extremely high repeat rate. This is possible by reason of the rapid recharging of the pulse losses. The magnetic stimulator 1 in accordance with the invention can achieve repeat rates with a frequency of 1000 Hz and higher. This offers the advantage that in this way clearly longer and more stable effects can be achieved during stimulation which are of relevance both during fundamental research and also in a therapeutic application. Strong lasting effects are a prerequisite for therapeutic success in a patient P.

FIG. 43 shows a normal muscle potential in order to illustrate effects which can be achieved by repetitive stimulation. The vertical arrows symbolise the magnitude of the effect, i.e. a rise means the increase in excitability and a fall means the decrease in excitability of the brain. The illustrated horizontal arrows show the duration of the effect which can be deduced at individual muscles of the patient P and which allows direct conclusions as to the change in excitability. The curve CTBS (continuous theta burst) shows the effect using a conventional protocol with a frequency of at most 50 Hz (T_{ISI}=20 ms). The two other curves shown in FIG. 43 show investigations carried out with the magnetic stimulator 1 in accordance with the invention, with so-called quattro pulses, which were carried out at a repeat rate of 200 Hz (t_{ISI}=5 ms) and a repeat rate of 20 Hz (t_{ISI}=50 ms). As shown by FIG. 43, the effect in the case of the high-frequency stimulation with the aid of the magnetic stimulator 1 in accordance with the invention is longer and more pronounced than with conventional stimulation. In FIG. 43, ‘Pre’ means the state prior to stimulation, while ‘Post’ means 1 to 4 min. after stimulation in a time range of 0 to 60 min.

The flexibility of the setting of the complex pulse patterns or pulse sequences PS is advantageous since an individual stimulation which adapts to the physiological characteristics of the subject or patient P is made possible. A specific example of individualised stimulation by means of magnetic stimulation is stimulation adapted to the so-called I-wave, which in conventional magnetic stimulators was possible only with two pulses, wherein the observed effects lasted only for a very short time. Adaptation of the application of the magnetic stimulation, in particular with a plurality of, in particular four to eight pulses, is of relevance for the effects achieved, which can thereby be markedly extended and are more pronounced. Furthermore, the current flow direction within the brain or the tissue, which is determined by the pulse polarity, also has a relevant influence.

FIG. 44 shows a diagram to illustrate the effect of a reversal in current flow (this corresponds to a change in polarity) in the stimulated brain, as is possible through the magnetic stimulator 1 in accordance with the invention. In FIG. 44, so-called I-wave-adapted stimulation with a frequency of 666 Hz is illustrated. AP means a current flow in the brain, which is generated by transcranial magnetic stimulation TMS and flows from front to rear. PA means a current flow flowing from the rear to the front. The horizontal arrows in FIG. 44 show the duration of the effect and vertical arrows show the level of the effect. In FIG. 44, it is possible to see a reversal in the effect of an increase to a decrease in the excitability of the brain when polarity is reversed. ‘Pre’ means a state prior to the intervention by means of high-frequency transcranial magnetic stimulation TMS. ‘Post’ means a state within 0 to 60 min. after start of the intervention.

Furthermore, after application of a double sinusoidal wave it is possible to prove experimentally the same effects with a still lower variability, as shown by FIG. 45. The brevity of these stimulation forms (ca. 2 min) make them practicable for investigation in young patients or children P. FIG. 45 shows the effects of a four-fold stimulation in the case of a double sinusoidal wave, which can be achieved with the magnetic stimulator 1 in accordance with the invention. An I-wave-adapted stimulation at a frequency of 666 Hz, i.e. a 1.5 ms interval between the four pulses, is likewise illustrated. The horizontal arrow shows the duration of the effect, the vertical arrow shows the level of the effect. FIG. 45 shows an extremely stable effect (increase in the excitability of the brain) with low variability even during measurement on only a few subjects P.

A further decisive advantage of the magnetic stimulator 1 in accordance with the invention with flexibly configurable pulse sequences is an individual adaptation of the pulse forms to the individual physiological characteristics of the patient P. For example, in children the so-called motor threshold, which represents a measure of the excitability of the brain at the stimulated site, is higher than in adult patients. In paediatric neurological diagnostics and in fundamental research, when using conventional magnetic stimulators, this frequently means that very young subjects can be investigated only to a limited extent.

FIG. 46 shows a diagram in which the motor threshold in the case of different pulse forms is illustrated. The pulses are applied to the brain in the current flow direction AP from the front to the rear (in the brain) or in the reverse current flow direction PA from the front to the rear. In FIG. 46, it is possible to see that the motor threshold of a pulse form, which is applied from front to rear (AP), is longer than pulses which are applied from rear to front (PA) or have a negative polarity.

The user-editor with the graphical interface used in the case of the magnetic stimulator 1 in accordance with the invention permits simple intuitive operation by the user and in particular a simple configuration of a pulse protocol or a complex pulse sequence PS. Furthermore, it is possible to carry out an automated adaptation to measured neurophysiological parameters by feedback of the parameters to the magnetic stimulator 1. By the use of the magnetic stimulator 1 a strongly reduced, interindividual variability of the protocols and stable induction of cortical plasticity with clear effects with respect to already existing conventional protocols can be achieved. These effective plasticity-inducing pulse protocols or pulse sequences PS permit therapeutic intervention on the patient P in order to optimise his/her neuronal plasticity, in particular in the case of neurological and psychiatric conditions. Furthermore, the magnetic stimulator 1 in accordance with the invention permits more extensive investigation of the human brain in order to obtain scientific knowledge.

1. A magnetic stimulator for stimulation of a tissue by a magnetic field having:
(a) a pulse generator device which comprises a pulse capacitor which can be charged by a charging circuit to generate a pulse sequence, consisting of pulses, with an adjustable repeat rate,
(b) wherein the pulse generator device comprises an oscillation circuit which contains the pulse capacitor and a stimulation coil, which is in a full bridge circuit connection with power switches to generate pulses, the pulse forms of which can be composed of pulse segments, wherein the power switches are each connected to a driver circuit which can be controlled by a programmable control device;
(b) wherein said programmable control device is adapted to adjust the pulse generator device in order to generate a complex pulse sequence which has individually configurable single pulses, wherein the generated complex pulse sequence is applied to said stimulation coil in order to generate the magnetic field, wherein the pulses are individually configurable with respect to their pulse form and polarity.

2. The magnetic stimulator as claimed in claim 1, wherein the complex pulse sequence output by the pulse generator device comprises pulse trains which each include pulse packets which each consist of a sequence of pulses, wherein a pulse form and/or polarity of the single pulses can be configured individually.

3. The magnetic stimulator as claimed in claim 1, wherein the programmable control device of the magnetic stimulator can be connected to a computer via an interface, on which computer a user-editor is provided to configure the pulse sequence.

4. The magnetic stimulator as claimed in claim 3, wherein the user-editor of the computer connected to the magnetic stimulator comprises a stimulus designer for configuration of a pulse form of the single pulses, a pulse packet assistant for configuration of at least one pulse packet consisting of single pulses and a pulse train assistant for configuration of at least one pulse train consisting of pulse packets.

5. The magnetic stimulator as claimed in claim 4,
wherein the pulse sequence configured by means of the user-editor is transmitted from the computer via the interface of the magnetic stimulator to the programmable control device of the magnetic stimulator and stored in a memory unit of the magnetic stimulator.

6. The magnetic stimulator as claimed in claim 1,
wherein the repeat rate of the pulse sequence indicates the number of pulses per second and can be adjusted within a range of 0 to 1 kHz.

7. The magnetic stimulator as claimed in claim 2,
wherein between pulse packets of the complex pulse sequence, which is generated by the pulse generator device of the magnetic stimulator, an evaluation pulse for measurement of a motor muscle response of the stimulated tissue is output.

8. The magnetic stimulator as claimed in claim 1,
wherein the pulse generator device pulse segment comprises a pulse segment rising in a sinusoidal manner, a constant pulse segment and a pulse segment descending in a sinusoidal manner.

9. (canceled)

10. The magnetic stimulator as claimed in claim 1,
wherein the pulse generator device of the magnetic stimulator has a charging circuit for recharging the pulse capacitor of the oscillation circuit with the adjusted repeat rate.

11. The magnetic stimulator as claimed in claim 10, wherein the charging circuit of the pulse generator device is a linear charging circuit which comprises a mains adapter for connection to a power supply network, an intermediate energy circuit for intermediate storage of the electrical energy supplied from the mains adapter and a charging regulator which is connected to the oscillation circuit of the pulse generator device.

12. The magnetic stimulator as claimed in claim 10, wherein the charging circuit of the pulse generator device is a clocked charging circuit which comprises
a mains adapter for connection to a power supply network,
a first DC/DC switching regulator for continuous operation,
an intermediate energy circuit for intermediate storage of the electrical energy supplied from the first DC/DC switching regulator, and
a second DC/DC switching regulator for pulsed operation, which is connected to the oscillation circuit of the pulse generator device.

13. The magnetic stimulator as claimed in claim 1,
wherein the pulse generator device comprises a coil monitoring circuit which monitors whether the stimulation coil is connected to the magnetic stimulator, and which comprises sensors for monitoring operating parameters of the stimulation coil.

14. The magnetic stimulator as claimed in claim 1,
wherein the programmable control device causes the pulse generator device to output the pulse sequence to the stimulation coil only after a system check of parameters of the magnetic stimulator has been successfully concluded.

15. The magnetic stimulator as claimed in claim 1,
wherein the programmable control device can be connected to a conducting electrode attached to the tissue to be stimulated in order to conduct a measurement signal and to generate a trigger signal.

16. The magnetic stimulator as claimed in claim 15, wherein the conducted measurement signal is evaluated by the programmable control device in order to determine a motor threshold.

17. (canceled)

18. (canceled)

19. (canceled)

20. (canceled)
